(19) United States
(12) Patent Application Publication
(10) Pub. No.: US 2018/0221449 A1
(43) Pub. Date: Aug. 9, 2018

(54) Compositions for Changing Body Composition, Methods of Use, and Methods of Treatment

(71) Applicants: Antonio E. CIVITARESE, Baton Rouge, LA (US); Frank L. GREENWAY, Baton Rouge, LA (US)

(72) Inventors: Antonio E. CIVITARESE, Baton Rouge, LA (US); Frank L. GREENWAY, Baton Rouge, LA (US)

(21) Appl. No.: 15/506,392

(22) PCT Filed: Aug. 25, 2015
(86) PCT No.: PCT/US15/46725

§ 371 Date: Feb. 24, 2017

Related U.S. Application Data
(60) Provisional application No. 62/041,223, filed on Aug. 25, 2014.
(60) Provisional application No. 62/147,168, filed on Apr. 14, 2015.

(51) Int. Cl. A61K 38/22; A61K 31/137; A61P 21/06; A61P 3/10; A61P 3/06

(52) U.S. Cl. CPC A61K 38/22; A61K 31/137; A61P 3/06; A61P 3/10; A61P 21/06

(57) ABSTRACT

The present disclosure provides compositions including an amylin receptor agonist and a beta-2-adreno-receptor agonist (e.g., an anti-hyperglycemia agent), compositions including at least two different anti-catabolic agents, compositions including at least two different anti-adiposity agents, methods of treating a condition (e.g., muscle wasting, muscle wasting-related condition, excess adiposity, an excess adiposity-related condition, and the like), methods of increasing muscle mass, formation of thermogenic brown adipose tissue (BAT), and/or decreasing white adipose tissue (WAT) content, methods of treating muscle wasting, methods of treating excess adiposity, and the like.

### CLAIM OF PRIORITY TO RELATED APPLICATION

This application claims priority to co-pending U.S. provisional application entitled “Compositions and Methods to Change Body Composition” having Ser. No. 62/147,168, filed on Apr. 14, 2015, which is entirely incorporated herein by reference. In addition, the application also claims priority to co-pending U.S. provisional application entitled “Compositions and Methods to Change Body Composition” having Ser. No. 62/041,223, filed on Aug. 25, 2014, which is entirely incorporated herein by reference.

### BACKGROUND

The epidemic of obesity and excess body fat accumulation is tightly linked to the prevalence of insulin resistance, type 2 diabetes, dyslipidemia, CVD and other disorders. The role of white adipose tissue (WAT) is to store lipids, however in syndromes such as obesity and insulin resistance, WAT adipose tissue can be a source of immune “dysfunction” and chronic inflammation that is linked to disease etiology. Brown adipose tissue (BAT) on the other hand is the opposite and its prime function is to bum off excess energy in the form of heat. In the BAT science area, this is termed uncoupling the energy transfer (of electrons) to make ATP (energy currency of the cell), to instead dissipate excess energy (adipose tissue) as heat via the uncoupling protein 1 (UCP1) molecule. Thus there is a need to reduce obesity and excess body fat, in particular WAT.

### SUMMARY

The present disclosure provides compositions including an amylin receptor agonist and a beta-2-adreno-receptor agonist (e.g., an anti-hyperglycemia agent), compositions including at least two different anti-catabolic agents, compositions including at least two different anti-adiposity agents, methods of treating a condition (e.g., muscle wasting, muscle wasting-related condition, excess adiposity, an excess adiposity-related condition, and the like), methods of increasing muscle mass, formation of thermogenic brown adipose tissue (BAT), and/or decreasing white adipose tissue (WAT) content, methods of treating muscle wasting, methods of treating excess adiposity, and the like.

An exemplary embodiment of the present disclosure includes a pharmaceutical composition, among others, that includes: a therapeutically effective amount of an amylin receptor agonist or a pharmaceutically acceptable salt thereof and a therapeutically effective amount of a beta-2-adreno-receptor agonist or a pharmaceutically acceptable salt thereof, and a pharmaceutically acceptable carrier, to treat a condition. In an embodiment, the condition can be selected from the group consisting of: muscle wasting, sarcopenia, a muscle wasting-related disorder, a muscle wasting-related disease, a muscle wasting-related condition, diabetes, insulin-resistance syndrome, cancer-cachexia, COPD, AIDS, congestive heart failure, sepsis, anorexia, pulmonary disease, excess adiposity, an excess adiposity-related disorder, an excess adiposity-related disease, an excess adiposity-related condition, lypodystrophy, nonalcoholic steatohepatitis, a cardiovascular disease, polycystic ovary syndrome, metabolic syndrome, and a combination thereof. In an embodiment, the condition can be used to increase muscle mass, reduce fat mass, or a combination thereof with or without an increase in endurance.

An exemplary embodiment of the present disclosure includes a method of treating a condition, among others, that includes: delivering to a subject in need thereof, a therapeutically effective amount of an amylin receptor agonist or a pharmaceutically acceptable salt thereof and a therapeutically effective amount of a beta-2-adreno-receptor agonist or a pharmaceutically acceptable salt thereof, and a pharmaceutically acceptable carrier.

An exemplary embodiment of the present disclosure includes a method of increasing muscle mass and formation of thermogenic brown adipose tissue (BAT), among others, that includes: delivering to a subject in need thereof, a therapeutically effective amount of an amylin receptor agonist or a pharmaceutically acceptable salt thereof and a therapeutically effective amount of a beta-2-adreno-receptor agonist or a pharmaceutically acceptable salt thereof, and a pharmaceutically acceptable carrier.

An exemplary embodiment of the present disclosure includes a method of decreasing white adipose tissue (WAT) content, among others, that includes: delivering to a subject in need thereof, a therapeutically effective amount of an amylin receptor agonist or a pharmaceutically acceptable salt thereof and a therapeutically effective amount of a beta-2-adreno-receptor agonist or a pharmaceutically acceptable salt thereof, and a pharmaceutically acceptable carrier.

An exemplary embodiment of the present disclosure includes a method of increasing muscle mass and formation of thermogenic brown adipose tissue (BAT), while simultaneously decreasing white adipose tissue (WAT) content, among others, that includes: delivering to a subject in need thereof, a therapeutically effective amount of an amylin receptor agonist or a pharmaceutically acceptable salt thereof and a therapeutically effective amount of a beta-2-adreno-receptor agonist or a pharmaceutically acceptable salt thereof, and a pharmaceutically acceptable carrier.

An exemplary embodiment of the present disclosure includes a pharmaceutical composition, among others, that includes: a therapeutically effective amount of at least two different anti-catabolic agents, and a pharmaceutically acceptable carrier, to treat a condition. In an embodiment, a first anti-catabolic agent can selected from the group consisting of a pramlintide acetate amylin analog agonist, and a long-acting amylin analog agonist; and wherein a second anti-catabolic agent can be selected from the group consisting of: a beta-2 receptor adreno-agonist, a beta-2 receptor adreno-agonist analog, a short acting beta-2 receptor adrenoagonist agonist, and a short acting adreno-agonist. In an embodiment, a first anti-catabolic agent can be selected from the group consisting of albuterol, an albuterol derivative, and an albuterol agonist; and wherein a second anti-catabolic agent can be selected from the group consisting of: clenbuterol, a clenbuterol derivative, and a clenbuterol agonist. In an embodiment, a first anti-catabolic agents can be selected from the group consisting of: a myostatin antagonist, an ActIIR-antagonist, an activin-A antagonist, IGF1-agonist, IGF1-receptor-agonist, IGF1, a recombinant IGF1R, IGF1 derivative, albuterol, clenbuterol, an albuterol analog, a clenbuterol analog, an albuterol agonist, a clenbuterol agonist, amylin, an amylin analog, and an amylin agonist. In an embodiment, a first anti-catabolic agent can be selected from the group consisting of: amylin, an amylin analog, and an amylin agonist, and a second anti-catabolic agent can be selected from the group consisting of: a beta-2 receptor adreno-agonist, an adreno-agonist, a beta-2 receptor adreno-agonist analog.

An exemplary embodiment of the present disclosure includes a method of treating muscle wasting, among others, that includes: delivering to a subject in need thereof, a therapeutically effective amount of at least two different anti-catabolic agents, and a pharmaceutically acceptable carrier.

An exemplary embodiment of the present disclosure includes a pharmaceutical composition, among others, that includes: a therapeutically effective amount of at least two different anti-adiposity agents, and a pharmaceutically acceptable carrier, to treat a condition.

An exemplary embodiment of the present disclosure includes a method of treating excess adiposity, among others, that includes: delivering to a subject in need thereof, a therapeutically effective amount of at least two different anti-adiposity agents, and a pharmaceutically acceptable carrier.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1A-B illustrate that mTOR Activation is enhanced by combination dosing.

FIG. 2 illustrates the combination therapy improves p70 86-Kinase-activation in Human RMS13 Cells.

FIG. 3 illustrates the combination dosing improves anabolic muscle biomarker profile in human RMS13 cells.

FIG. 4 illustrates that insulin provides minimal improvement on catabolic biomarkers.

FIG. 5 illustrates that the combination therapy directly lowers myostatin secretion in human RMS13 cell culture.

FIG. 6 illustrates that that pramlintide targets anabolic specificity and calcitonin does not regulate myostatin mRNA in RMS13 cells.

FIG. 7 illustrates that the combination therapy improves body composition in healthy-young B6 mice after 8-weeks.

FIG. 8 illustrates the cumulative food intake and % body fat change after 8 wk. of treatment.

FIG. 9 illustrates that the combination therapy increases muscle tissue.

FIG. 10 illustrates the combination therapy improves fasting glucose (IPGTT).

FIG. 11 illustrates that the combination therapy improves body composition in healthy-young B6 mice (20 wk.).

FIG. 12 illustrates the combination therapy increases exercise endurance in young mice.

FIG. 13 illustrates that the combination therapy improves body composition in elderly B6 Mice (20 wk.).

FIG. 14 illustrates that combination therapy does not improve exercise endurance in elderly mice.

FIG. 15 illustrates that the combination treatment induces a lean phenotype in mice.

FIG. 16 illustrates that the Brown Adipose Tissue (BAT) mass (g) is dramatically enhanced after 8 wk. of combination treatment—in vivo Study 1.

FIG. 17 illustrates that the UCP1 expression in BAT is dramatically induced after 8 weeks of combination therapy in mice.

FIG. 18 illustrates that the PRDM16 expression in BAT is induced after 8 weeks of combination therapy in mice.

FIG. 19 illustrates the combination therapy differentially alters metabolic biomarkers between young vs. old animals in response to cold exposure.

FIG. 20 illustrates that the combination therapy increases energy expenditure independent of changes in body weight and activity levels.

### DISCUSSION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions

Unless otherwise defined, all technical and scientific terms used herein have the same meaning as commonly understood by one of ordinary skill in the art of molecular biology, medicinal chemistry, and/or organic chemistry. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

The term “unit dosage form” as used herein, refers to physically discrete units suitable as unitary dosages for human and/or animal subjects, each unit containing a predetermined quantity of a compound (e.g., compositions or pharmaceutical compositions, as described herein) calculated in an amount sufficient to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound in the subject.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used in the specification and claims includes one and more such excipients, diluents, carriers, and adjuvants.

As used herein, a “pharmaceutical composition” is meant to encompass a composition or pharmaceutical composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

The term “therapeutically effective amount” as used herein refers to that amount of an embodiment of the composition or pharmaceutical composition being administered that will relieve to some extent one or more of the symptoms of a condition being treated, and/or that amount that will prevent, to some extent, one or more of the symptoms of the condition, that the subject being treated has or is at risk of developing.

“Pharmaceutically acceptable salt” refers to those salts that retain the biological effectiveness and optionally other properties of the free bases and that are obtained by reaction with inorganic or organic acids such as hydrochloric acid, hydrobromic acid, sulfuric acid, nitric acid, phosphoric acid, methane sulfonic acid, ethane sulfonic acid, p-toluene sulfonic acid, salicylic acid, malic acid, maleic acid, succinic acid, tartaric acid, citric acid, and the like.

In the event that embodiments of the disclosed compounds (agents) in the composition or pharmaceutical composition form salts, these salts are within the scope of the present disclosure. Reference to a compound used in the composition or pharmaceutical composition of any of the formulas herein is understood to include reference to salts thereof, unless otherwise indicated. The term “salt(s)”, as employed herein, denotes acidic and/or basic salts formed with inorganic and/or organic acids and bases. In addition, when a compound contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of a compound may be formed, for example, by reacting the compound with an amount of acid or base, such as an equivalent amount, in a medium such as one in which the salt precipitates or in an aqueous medium followed by lyophilization.

Embodiments of the compounds of the composition or pharmaceutical composition of the present disclosure that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the compounds (agents) of the composition or pharmaceutical composition of the present disclosure that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds (agents) of the composition or pharmaceutical composition of the present disclosure are also contemplated herein.

To the extent that the disclosed the compounds (agents) of the composition or pharmaceutical composition of the present disclosure, and salts thereof, may exist in their tautomeric form, all such tautomeric forms are contemplated herein as part of the present disclosure.

All stereoisomers of the compounds (agents) of the composition or pharmaceutical composition of the present disclosure, such as those that may exist due to asymmetric carbons on the various substituents, including enantiomeric forms (which may exist even in the absence of asymmetric carbons) and diastereomeric forms are contemplated within the scope of this disclosure. Individual stereoisomers of the compounds of the disclosure may, for example, be substantially free of other isomers, or may be admixed, for example, as racemates or with all other, or other selected, stereoisomers. The stereogenic centers of the compounds of the present disclosure can have the S or R configuration as defined by the IUPAC 1974 Recommendations.

The term “prodrug” refers to an inactive precursor of the compounds (agents) of the composition or pharmaceutical composition of the present disclosure that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N. J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS Pharm Sci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

The term “administration” refers to introducing a composition (agents) of the present disclosure into a subject. One preferred route of administration of the composition is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, “treat”, “treatment”, “treating”, and the like refer to acting upon a condition, a disease or a disorder with a composition (agents) to affect the condition, disease or disorder by improving or altering it. The improvement or alteration may include an improvement in symptoms or an alteration in the physiologic pathways associated with the condition, disease, or disorder or the loss of fat and/or increase in muscle mass. “Treatment,” as used herein, covers one or more treatments of a condition or a disease in a subject (e.g., a mammal, typically a human or non-human animal of veterinary interest), and includes: (a) reducing the risk of occurrence of the condition in a subject determined to be predisposed to the condition or disease but not yet diagnosed with it (b) impeding the development of the condition, (c) relieving the condition, e.g., causing regression of the condition and/or relieving one or more condition symptoms, and (d) decreasing fat and/or increasing muscle mass.

As used herein, the terms “prophylactically treat” or “prophylactically treating” refers completely or partially preventing (e.g., about 50% or more, about 60% or more, about 70% or more, about 80% or more, about 90% or more, about 95% or more, or about 99% or more) a condition, a disease, or a symptom thereof and/or may be therapeutic in terms of a partial or complete cure for a condition, and/or adverse effect attributable to the condition.

As used herein, the term “subject,” or “patient,” includes humans, mammals (e.g., mice, rats, pigs, cats, dogs, and horses), and ayes. Typical subjects to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

### Discussion

The present disclosure provides compositions including an amylin receptor agonist and a beta-2-adreno-receptor agonist (e.g., an anti-hyperglycemia agent), compositions including at least two different anti-catabolic agents, compositions including at least two different anti-adiposity agents, methods of treating a condition (e.g., muscle wasting, muscle wasting-related condition, excess adiposity, an excess adiposity-related condition, and the like), methods of increasing muscle mass, formation of thermogenic brown adipose tissue (BAT), and/or decreasing white adipose tissue (WAT) content, methods of treating muscle wasting, methods of treating excess adiposity, and the like.

The epidemic of obesity and excess body fat accumulation is tightly linked to the prevalence of insulin resistance, type 2 diabetes, dyslipidemia, CVD and other disorders. The role of white adipose tissue (WAT) is to store lipids, however in syndromes such as obesity and insulin resistance, WAT adipose tissue can be a source of immune “dysfunction” and chronic inflammation that is linked to disease etiology. Brown adipose tissue (BAT) on the other hand is the opposite and its prime function is to bum off excess energy in the form of heat. In the BAT science area, this is termed uncoupling the energy transfer (of electrons) to make ATP (energy currency of the cell), to instead dissipate excess energy (adipose tissue) as heat via the uncoupling protein 1 (UCP1) molecule.

Embodiments of the present disclosure can include administering a composition including one or more agents (e.g., amylin and/or beta-2 receptor agonist) to a subject. By doing this one or more of the following can be achieved: synergistically lower excess body fat relative to non-treatment by specifically targeting the fat cell; synergistically preserve lean body mass by inducing anabolic pathways to target muscle building, induce muscle building targets in skeletal muscle (PGC1a, mTFAM, IGF1R, mTOR activity) and lower the level of catabolic targets (MSTN, Activin Recptor Iib/Ib, Foxo1/3 Smad3); specially target HSL by increasing activity and flow of free fatty acids from WAT and lowering LPL-activity; and slow the rate of re-uptake into WAT, which allows for target organs such as skeletal muscle and BAT to use FFA as fuel and in turn lowering excess body fat; synergistically increase the formation of beige and brown adipocytes; and synergistically increase the expression of key BAT factors (UCP1, PGC1a, Cidea, PRDM16, Pax3). As a result, composition and methods of treatment of the present disclosure can be used to induce or increase lean body mass, increase brown adipose tissue formation/UCP1 content, and improve body composition profile by lowering adiposity while maintaining muscle mass or increasing muscle mass and be used for the prevention of excess adiposity, metabolic disease and its' co-morbidities.

In general, embodiments of the present disclosure can be used to control, treat, and/or prevent a condition in a subject, where the condition (e.g., which can include a condition, disease, or disorder) can include: muscle wasting, sarcopenia, a muscle wasting-related disorder, a muscle wasting-related disease, a muscle wasting-related condition, diabetes, insulin-resistance syndrome, cancer-cachexia, COPD, AIDS, congestive heart failure, sepsis, anorexia, pulmonary disease, excess adiposity, an excess adiposity-related disorder, an excess adiposity-related disease, an excess adiposity-related condition, lypodystrophy, nonalcoholic steatohepatitis, a cardiovascular disease, polycystic ovary syndrome, metabolic syndrome, and a combination thereof.

In an embodiment the condition can be treated using a composition (e.g., a pharmaceutical composition) using one or more agents, where the agent can include anti-catabolic agents, anti-adiposity agents, amylin receptor agonists, beta-2-adreno-receptor agonists, any combination of these types of agents (e.g., an amylin receptor agonist and a beta-2-adreno-receptor agonist), any combination of compounds within each type of agent provided herein (e.g., two anti-catabolic agents). In an embodiment, the agents can be included in a single composition (e.g., a pharmaceutical composition), or administered individually (e.g., simultaneous, concurrent, or sequential administration)

In a particular embodiment, the present disclosure provides for where the condition is a situation where it is desirable to increase muscle mass, reduce fat mass, or a combination thereof with or without an increase in endurance. For example, this type of condition could be used for cosmetic purposes or for the treatment of pediatric obesity, where there are advantages to decreasing fat loss without having to limit food intake in people with growing brains. In an another example, this type of condition may apply for soldiers, sailors, marines, air force personnel, police officers, firefighters, body builders, athletes, or the like, where increased muscle mass, reduced fat mass and increased physical endurance are advantageous.

In a particular embodiment, the present disclosure provides for methods for treating a subject suffering from muscle wasting, sarcopenia, muscle wasting-related disorder, a muscle wasting-related disease, a muscle wasting-related condition, diabetes, insulin-resistance syndrome, cancer-cachexia, COPD, AIDS, congestive heart failure, sepsis, anorexia, pulmonary disease, disuse, or a desire to gain body weight. In particular, the method includes delivering to a subject in need a therapeutically effective amount of at least two different agents disclosed herein and, optionally, a pharmaceutically acceptable carrier. In an embodiment, administration of the agents can result in a synergistic effect as compared to the sum of administering the agents separately for treating one or more conditions.

In another embodiment, the present disclosure provides for methods for treating a subject suffering from excess adiposity, an excess adiposity-related disorder, an excess adiposity-related disease, an excess adiposity-related condition, diabetes, insulin-resistance syndrome, lypodystrophy, nonalcoholic steatohepatitis, a cardiovascular disease, polycystic ovary syndrome, metabolic syndrome, or a desire to lose body fat, or a desire to lose weight. In particular, the method includes delivering to a subject in need a therapeutically effective amount of at least two different agents disclosed herein and, optionally, a pharmaceutically acceptable carrier. In an embodiment, administration of the agents can result in a synergistic effect as compared to the sum of administering the agents separately for treating one or more conditions.

In an embodiment, the composition or pharmaceutical composition can include a therapeutically effective amount of an amylin receptor agonist or a pharmaceutically acceptable salt thereof and a therapeutically effective amount of a beta-2-adreno-receptor agonist or a pharmaceutically acceptable salt thereof (e.g., optionally a pharmaceutically acceptable carrier) to treat a condition, such as those described herein. In an embodiment, the amount of each agent can be selected based on the individual and the specific goals for fat lose and/or muscle gain or maintaining a certain muscle mass, where the amount of each agent can depend upon the age, health, body composition, other medications, and goals to be achieved. The ratio of each agent can be about 1:99 to 99:1 or about 1:1 (amylin receptor agonist:beta-2-adreno-receptor agonist).

In an embodiment, the amylin receptor agonist can be an amylin-hormone mimetic. The amylin-hormone mimetic can include a compound such as pramlintide. In addition, amylin peptides that can be used in embodiments of the present disclosure are described in U.S. Pat. Nos. 7,928,060, 8,114,958, 8,378,067, 8,486,890, 8,575,090, 8,575,091, 8,598,120, and Patent Applications having Publication Numbers 20120196796 and 2014221287, each of which is incorporated herein by reference for the amylin receptor agonist disclosed.

In an embodiment, the beta-2-adreno-receptor agonist can include albuterol, an albuterol derivative, an albuterol agonist, and a combination thereof. In an embodiment, the albuterol derivative or agonist can include terbutaline, levalbuterol, pirbuterol, procaterol, clenbuterol, metaproterenol, fenoterol, bitolterol mesylate, ritodrine, isprenaline, salmererol, fomoterol, bambuterol, clenbuterol, olodaterol, indacaterol, and calcitonins.

Embodiments of the present disclosure can be used to control, treat, and/or prevent muscle wasting, excess adiposity, obesity-related conditions (e.g., disorders, diseases, and the like) or build muscle and endurance. In a particular embodiment, the method can be used to increase muscle mass, form of thermogenic BAT, and/or decrease white adipose tissue (WAT) content. In an embodiment, the method can include administration of a combination compounds such as at least one an amylin receptor agonist, at least one beta-2-adreno-receptor agonist, at least one anti-catabolic agent, at least one anti-adiposity agents, where the compounds can be included in a single composition (e.g., a pharmaceutical composition), or administered individually (e.g., simultaneous, concurrent, or sequential administration).

In a particular embodiment, the method can include administration of a combination compounds that can increase muscle mass (e.g., by about 1 to 25% relative to the initial muscle mass), increase formation of thermogenic brown adipose tissue (BAT) (e.g., by about 1 to 20% relative to the initial thermogenic BAT), and/or decrease white adipose tissue (WAT) content (e.g., by about 1 to 20% relative to the initial WAT content), where the compounds can be included in a single composition (e.g., a pharmaceutical composition), or administered individually (e.g., simultaneous, concurrent, or sequential administration).

An embodiment provides for methods whereby the subject reduces body fat (e.g., by at least 10% relative to the starting body fat, by at least 15% relative to the starting body fat, by at least 20% relative to the starting body fat) and/or the subject loses ectopic fat. In an embodiment, the method can include administration of a combination compounds that can reduce weight of a subject associated with WAT by lowering the excess composition of WAT.

An embodiment provides for methods whereby the subject can increase lean muscle mass (e.g., by at least about 5% relative to starting muscle mass, by at least about 10% relative to starting muscle mass, or by at least about 15% relative to starting muscle mass) and/or improve functional performance of the subject as it pertains to musculature.

In an embodiment, the present disclosure is directed to controlling, changing, or treating a subject having an excess adiposity-to-muscle ratio and excess-adiposity-to-muscle ratio-related conditions. In an embodiment, the present disclosure can be used to reduce the adiposity-to-muscle ratio by about 1 to 25% relative to the starting adiposity-to-muscle ratio.

In an embodiment, the composition or pharmaceutical composition can include a therapeutically effective amount of at least two different anti-catabolic agents or a pharmaceutically acceptable salt of each one or a pharmaceutically acceptable salt thereof (e.g., optionally a pharmaceutically acceptable carrier) to treat a muscle wasting condition. In an embodiment, the amount of each agent can be selected based on the individual and the specific goals to treat muscle wasting, where the amount of each agent can depend upon the age, health, body composition, other medications, and goals to be achieved. The ratio of each agent can be about 1:99 to 99:1 or about 1:1 (first anti-catabolic agent:second anti-catabolic agent).

The phrase “muscle wasting” refers to a decrease in muscle mass that often occurs with disuse such as with bed rest in a hospital or with aging or associated some conditions or diseases or treatments for those conditions or diseases.

In an embodiment, a first anti-catabolic agent can be pramlintide acetate, amylin analog agonist, or a long-acting amylin analog agonist, while a second anti-catabolic agent is not pramlintide acetate, amylin analog agonist, or a long-acting amylin analog agonist. In an embodiment, the first anti-catabolic agent can include terbutaline, levalbuterol, pirbuterol, procaterol, clenbuterol, metaproterenol, fenoterol, bitolterol mesylate, ritodrine, isprenaline, salmererol, fomoterol, bambuterol, clenbuterol, olodaterol, indacaterol, and calcitonins.

In an embodiment, the second anti-catabolic agent can be a beta-2 receptor adreno-agonist, a beta-2 receptor adreno-agonist analog, a beta-2 receptor adreno-agonist (short acting), or a beta-2 receptor adreno-agonist analog (short acting). In an embodiment, the beta-2 receptor adreno-agonist can include terbutaline, levalbuterol, pirbuterol, procaterol, clenbuterol, metaproterenol, fenoterol, bitolterol mesylate, ritodrine, isprenaline, salmererol, fomoterol, bambuterol, clenbuterol, olodaterol, indacaterol, and calcitonins.

In an embodiment, the first anti-catabolic agent can be albuterol, an albuterol derivative, or an albuterol agonist, while the second anti-catabolic agent is clenbuterol, a clenbuterol derivative, or a clenbuterol agonist. In an embodiment, the albuterol derivative or agonist can include terbutaline, levalbuterol, pirbuterol, procaterol, metaproterenol, fenoterol, bitolterol mesylate, ritodrine, isprenaline, salmererol, fomoterol, bambuterol, clenbuterol, olodaterol, indacaterol, and calcitonins. In an embodiment, the clenbuterol derivative or agonist can include terbutaline, levalbuterol, pirbuterol, procaterol, metaproterenol, fenoterol, bitolterol mesylate, ritodrine, isprenaline, salmererol, fomoterol, bambuterol, clenbuterol, olodaterol, indacaterol, and calcitonins.

In an embodiment, the composition can include and the method contemplates administering of at least two different anti-catabolic agents, where a first anti-catabolic agent is selected from a myostatin antagonist, an ActIIR-antagonist, an activin-A antagonist, IGF1-agonist, IGF1-receptor-agonist, IGF1, a recombinant IGF1R, IGF1 derivative, albuterol, clenbuterol, an albuterol analog, a clenbuterol analog, an albuterol agonist, a clenbuterol agonist, amylin, an amylin analog, and an amylin agonist, and where the second anti-catabolic agent can be beta-2 receptor adreno-agonist, a peptide-based beta-2 receptor adreno-agonist, a beta-2 receptor adreno-agonist analog, or a peptide-based beta-2 receptor adreno-agonist analog. The albuterol analog, the clenbuterol analog, the albuterol agonist, the clenbuterol agonist, the amylin analog, and the amylin agonist can includes those compounds described herein. In an embodiment, the amount of each agent can be selected based on the individual and the specific goals to treat muscle wasting, where the amount of each agent can depend upon the age, health, body composition, other medications, and goals to be achieved. The ratio of each agent can be about 1:99 to 99:1 or about 1:1 (first anti-catabolic agent:second anti-catabolic agent).

In an embodiment, a first anti-catabolic agent can include amylin, an amylin analog or an amylin agonist in combination with a second anti-catabolic agent can be a beta-2 receptor adreno-agonist, a peptide-based beta-2 receptor adreno-agonist, a beta-2 receptor adreno-agonist analog, or a peptide-based beta-2 receptor adreno-agonist analog. The beta-2 receptor adreno-agonist, beta-2 receptor adreno-agonist analog, amylin analog and the amylin agonist can includes those compounds described herein. In an embodiment, the amount of each agent can be selected based on the individual and the specific goals to treat muscle wasting, where the amount of each agent can depend upon the age, health, body composition, other medications, and goals to be achieved. The ratio of each agent can be about 1:99 to 99:1 or about 1:1 (first anti-catabolic agent:second anti-catabolic agent).

Embodiments of the present disclosure provide for methods for treating muscle wasting in a subject. In particular, the method includes delivering to a subject in need a therapeutically effective amount of at least two different anti-catabolic agents and a pharmaceutically acceptable carrier. In an embodiment, administration of the anti-catabolic agents can result in a synergistic effect as compared to the sum of administering the agents separately. In an embodiment, at least one anti-catabolic agent acts upon structures in the skeletal muscle to increase protein synthesis via mTOR activation or anti-catabolic-muscle breakdown by inhibiting pathways associated with protein breakdown at the level of FoxO-transcription factors. In an embodiment, at least one anabolic agent that acts upon structures in muscle structures involved in ActIIR-activity or direct suppression of myostatin transcription and secretion.

In an embodiment, the composition or pharmaceutical composition can include a therapeutically effective amount of at least two different anti-adiposity agents or a pharmaceutically acceptable salt of each one or a pharmaceutically acceptable salt thereof (e.g., optionally a pharmaceutically acceptable carrier) to treat a excess adiposity condition. In an embodiment, the amount of each agent can be selected based on the individual and the specific goals to treat muscle wasting, where the amount of each agent can depend upon the age, health, body composition, other medications, and goals to be achieved. The ratio of each agent can be 1:99 to 99:1 (first anti-adiposity agent:second anti-adiposity agent).

The phrase “excess adiposity” in humans refers to excess adiposity of about 30% or more in females and about 25% or more in males, but can depend upon age, height, muscle mass, and other conditions.

In an embodiment the first anti-adiposity can be amylin, an amylin analog, or an amylin agonist, while the second anti-adiposity agent can be a beta-2 receptor adreno-agonist, a peptide-based beta-2 receptor adreno-agonist, a beta-2 receptor adreno-agonist analog, or a peptide-based beta-2 receptor adreno-agonist. The agents having the same name as those described herein have the same meaning and include the same compounds.

Embodiments of the present disclosure provide for methods for treating excess adiposity in a subject. In particular, the method includes delivering to a subject in need a therapeutically effective amount of at least two different anti-adiposity agents and a pharmaceutically acceptable carrier. In an embodiment, administration of the anti-adiposity agents can result in a synergistic effect as compared to the sum of administering the agents separately.

In an embodiment, the methods includes of treating excess adiposity by administering a combination of a first anti-adiposity agent and a second anti-adiposity. In an embodiment the first anti-adiposity can be amylin, an amylin analog, or an amylin agonist, while the second anti-adiposity agent can be a beta-2 receptor adreno-agonist, a peptide-based beta-2 receptor adreno-agonist, a beta-2 receptor adreno-agonist analog, or a peptide-based beta-2 receptor adreno-agonist, where specific examples of each are provided herein.

It should be noted that the therapeutically effective amount to result in uptake of the composition(s) or pharmaceutical composition(s) (e.g., including the agent or combination of agents) into the subject can depend upon a variety of factors, including for example, the age, body weight, general health, sex, and diet of the subject; the time of administration; the route of administration; the rate of excretion of the specific compound employed; the duration of the treatment; the existence of other drugs used in combination or coincidental with the specific composition employed; the type(s) of bacteria; and like factors well known in the medical arts.

### Pharmaceutical Formulations and Routes of Administration

Embodiments of the present disclosure include an agent or combination of agents as identified herein and formulated with one or more pharmaceutically acceptable excipients, diluents, carriers and/or adjuvants. In addition, embodiments of the present disclosure include an agent or combination of agents formulated with one or more pharmaceutically acceptable auxiliary substances. In particular an agent or combination of agents can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the agent or combination of agents can be administered to the subject using any means capable of resulting in the desired effect. Thus, the agent or combination of agents can be incorporated into a variety of formulations for therapeutic administration. For example, the agent or combination of agents can be formulated into pharmaceutical compositions by combination with appropriate, pharmaceutically acceptable carriers or diluents, and may be formulated into preparations in solid, semisolid, liquid or gaseous forms, such as tablets, capsules, powders, granules, ointments, solutions, suppositories, injections, inhalants and aerosols.

In pharmaceutical dosage forms, the agent or combination of agents may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the agent or combination of agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the agent or combination of agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the agent or combination of agents can be utilized in aerosol formulation to be administered via inhalation. Embodiments of the agent or combination of agents can be formulated into pressurized acceptable propellants such as dichlorodifluoromethane, propane, nitrogen and the like.

Furthermore, embodiments of the agent or combination of agents can be made into suppositories by mixing with a variety of bases such as emulsifying bases or watersoluble bases. Embodiments of the agent or combination of agents can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the agent or combination of agents in a composition as a solution in sterile water, normal saline or another pharmaceutically acceptable carrier.

Embodiments of the agent or combination of agents can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles in accordance with the present disclosure.

In an embodiment, the agent or combination of agents can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the agent or combination of agents can be accomplished using any of a variety of refillable, pump systems. Pumps provide consistent, controlled release over time. In some embodiments, the agent or combination of agents can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a sub-dermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent (e.g., the agent or combination of agents) can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the agent or combination of agents are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the agent or combination of agents adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, the agent or combination of agents may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the agent or combination of agents described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Dosages

Embodiments of the agent or combination of agents can be administered to a subject in one or more doses. Those of skill will readily appreciate that dose levels can vary as a function of the specific the agent or combination of agents administered, the severity of the symptoms and the susceptibility of the subject to side effects. Preferred dosages for a given compound are readily determinable by those of skill in the art by a variety of means.

In an embodiment, multiple doses of the agent or combination of agents are administered. The frequency of administration of the agent or combination of agents can vary depending on any of a variety of factors, e.g., severity of the symptoms, and the like. For example, in an embodiment, the agent or combination of agents can be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), or four times a day, but will depend upon the specific agents, age, condition, and the like. As discussed above, in an embodiment, the agent or combination of agents is administered continuously.

The duration of administration of the agent or combination of agents analogue, e.g., the period of time over which the agent or combination of agents is administered, can vary, depending on any of a variety of factors, e.g., patient response, etc. For example, the agent or combination of agents in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

### Routes of Administration

Embodiments of the present disclosure provide methods and compositions for the administration of the active agent (e.g., the agent or combination of agents) to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An active agent (e.g., the agent or combination of agents) can be administered in a single dose or in multiple doses.

Embodiments of the agent or combination of agents can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the agent or combination of agents. Where systemic delivery is desired, administration typically involves invasive or systemically absorbed topical or mucosal administration of pharmaceutical preparations.

In an embodiment, the agent or combination of agents can also be delivered to the subject by enteral administration. Enteral routes of administration include, but are not limited to, oral and rectal (e.g., using a suppository) delivery.

Methods of administration of the agent or combination of agents through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1—In Vitro M.O.A. in Human Muscle using a Combination Approach

We first examined the association between a pramlintide and albuterol formulation (single and combination dosing) on the activity of the mammalian target of rapamycin (mTOR) in a human myoblast cell model derived from RMS13 cells (purchased from ATCC, Manassas, Va., USA). mTOR is a highly conserved kinase that exists in two functional complexes, TOR complex 1 (TORC1) and TOR complex 2 (TORC2), that are conserved from yeast to mammals. Given its high conservation across species, mTOR serves as a key biomarker for protein synthesis. mTOR (TORC1 and TORC 2) is a master kinase that is crucial for cell growth and is a chief enzyme in protein synthesis (Otulakowski et al., 2007). Determining optimal ratios and concentrations of each drug (in single or combination) was an important first step to guide the mechanism of action biomarker studies. This initial study was undertaken for 24 and 48 hours; only the 24-hour results are shown (n=6 each group).

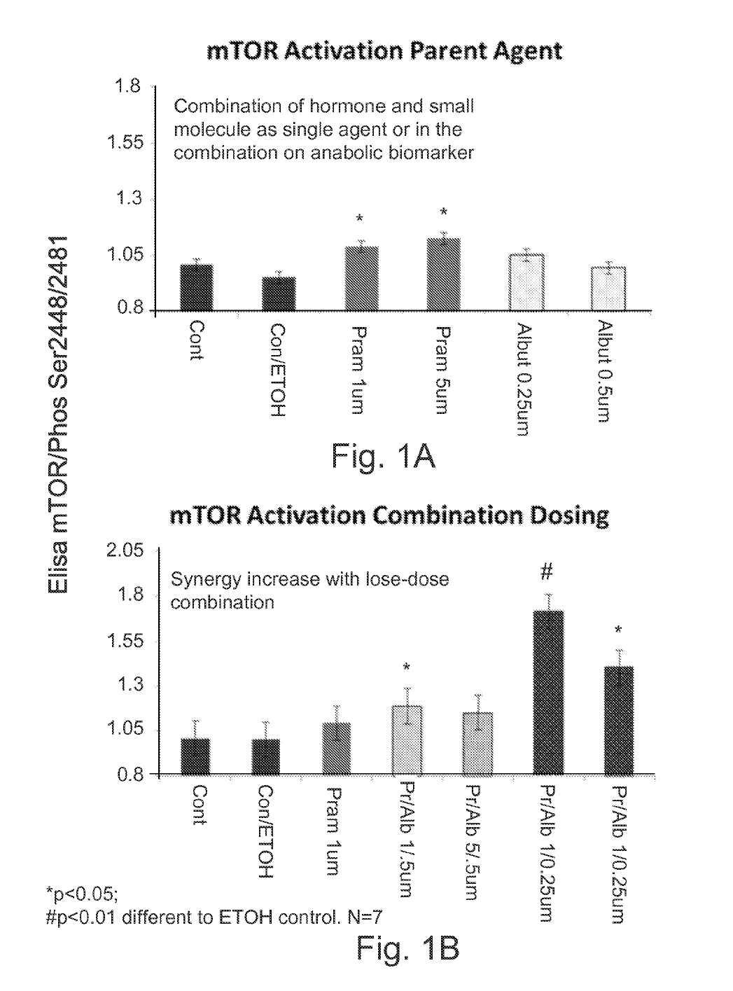

Using a single-drug dose (FIG. 1—top panel), pramlintide alone induced mTOR activation at 1 and 5 mm (both p<0.05). However, when low-dose pramlintide (1 mm) was combined with low (0.25 mm) and -mid (0.5 mm) dose albuterol, we observed a more than additive induction in mTOR activation (both p<0.01). The greatest increase was observed at the lowest concentration of pramlintide/albuterol (P/A; 1 mm/0.25 mm), suggesting an in vitro “dose-sparing” pharmacology with the combined dosing.

Example 2—Biomarker Combination Results Confirmed with S6-Kinase Assay

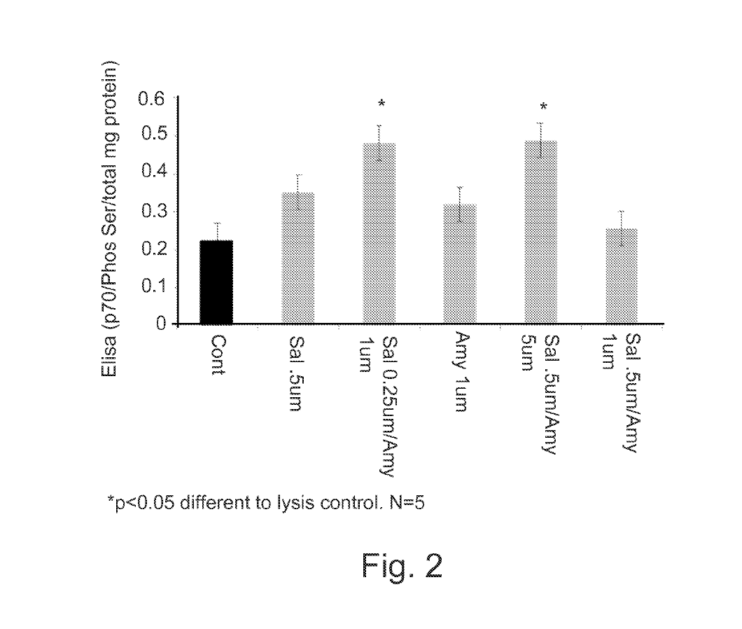

Protein synthesis and degradation are dynamically regulated processes that act in concert to control the accretion or loss of muscle mass. The vast majority of mechanisms known to regulate protein synthesis involve modulation of the initiation phase of mRNA translation, which comprises a series of reactions that result in the binding of initiator methionyl-tRNA and mRNA to the 40S ribosomal subunit. Raptor is a 150 kDa mTOR-binding protein that also binds S6K, serves as a scaffold protein of mTOR and facilitates mTOR phosphorylation of S6K (Magnuson et al., 2012; Nojima et al., 2003). S6K has been implicated as an important positive regulator of biological processes, such as cell growth, proliferation, and regulating ribosomal activity and protein synthesis (Holz and Blenis, 2005; Magnuson et al., 2012; Nojima et al., 2003; Tremblay and Marette, 2001; Zito et al., 2007). Consistent with results in FIG. 1, combination dosing using P/A; 1 mm/0.25 um, increased SK6 activity to a greater extent than any of the single-arm parent agents (FIG. 2, p<0.05, n=5 replicates).

### Example 3—Combination Dosing Improves Anabolic Muscle Biomarker Profile in Human RMS13 Cells

Recent findings have highlighted a complex scenario wherein an intricate network of signaling pathways regulates the size of myofibers and the contractile performance of muscle. These different pathways crosstalk and modulate one another at different levels, coordinating protein synthesis and degradation simultaneously. Highlighted here is the involvement of the major pathway (IGF1-Akt-FoxO, myostatin) in muscle atrophy/building and how using these biomarkers can improve the understanding how a combination therapy works to promote muscle-building.

Insulin-like growth factor 1 (IGF-1), is a circulating growth factor that binds to the IGF1-receptor (IGF1R), and is also produced locally by many tissues, including skeletal muscle (reviewed in (Schiaffino, 2010). Muscle-specific overexpression of a locally acting IGF-1 isoform in mice showed that localized IGF-1 expression sustains muscle growth and regeneration (Musaro et al., 2001). Indeed, these mice have muscle hypertrophy and display higher regenerative capacity and maintenance of muscle mass during aging (Musaro et al., 2001). Other studies have shown that IGF-1 and/or insulin signaling can suppress protein breakdown while promoting muscle growth (Rommel et al., 2001; Sacheck et al., 2004). IGF-1 transgenic mice are resistant to muscle atrophy induced by angiotensin treatment, and to cardiac cachexia (Schulze et al., 2005). IGF-1 is a principle regulator of cellular growth, although its use as a therapeutic target is limited due its tumorigenic potential. Activating this system vis-a-vis secondary cascades may be an alternative method to circumvent the potential link to oncology. Pramlintide therapy in combination with beta-2-agonist is a therapeutic alternative. Studies demonstrate that both amylin and IGF-1 are able to induce osteoblast proliferation and growth. The inhibition of the amylin receptor in osteoblast abolishes IGF-1's ability to promote bone growth (Cornish et al., 1998; Cornish et al., 1999). Similarly, pharmacological inhibition of the amylin receptor abolishes IGF1-induced cell proliferation. Collectively, these data indicate a functional relationship between IGF-1 signaling and amylin biology, and may represent an alternative activation mechanism for IGF-1 activity and protein synthesis that should have no tumorigenic potential.

The precise timing of the removal of proteins regulating transcription or metabolic pathways is required to avoid jeopardizing the organism's survival. Thus, most intracellular proteins are degraded by the same ATP-dependent, ubiquitin-proteasome system (UPS). UPS-mediated degradation of specific proteins is regulated at several steps. First, proteins are marked by a conjugation to ubiquitin (Ub) via an ATP-dependent process requiring three enzymatic reactions. The first two reactions are catalyzed by a single, E1 Ub-activating enzyme, secondly by E2 Ub-carrier proteins and lastly by more than 1000 E3 ubiquitin ligases. In muscle wasting conditions, the two main E3 ligases regulating the UPS breakdown of protein are Atrogin-1 (also called MAFbx) and MuRF1 (for muscle ring finger type) because they recognize specific muscle proteins (Sandri, 2002, 2008; Sandri et al., 2006; Sartori et al., 2009). Not surprisingly, there is an increase in Atrogin-1/MAFbx and MuRF1 expression in many catabolic conditions including cancer, diabetes, chronic kidney disease, starvation and denervation (Sandri, 2002, 2008; Sandri et al., 2006; Sartori et al., 2009). Akt controls protein degradation, via turning off the transcription factors Foxo-1 and -3. Akt phosphorylates FoxO proteins, promoting their export from the nucleus to the cytoplasm, where they are stripped of their transcriptional activity. The reduced activity of the Akt pathway observed in different models of muscle atrophy and insulin resistance results in decreased levels of phosphorylated FoxO in the cytoplasm and a marked increase of nuclear FoxO activity that increases protein break down via UPS (reviewed in (Calnan and Brunet, 2008)). The translocation and activity of FoxO-1 and -3 is required for the up regulation of Atrogin-1 and MuRF1, and FoxO-3 is sufficient to promote Atrogin-1 expression and muscle atrophy when transfected into skeletal muscles in vivo (Sandri, 2008).

FoxO activity is also modulated by direct or indirect actions of cofactors and by interacting with other transcription factors. FoxO has been found to interact with PGC1a, a critical cofactor for mitochondrial biogenesis (Puigserver et al., 2003). Maintaining high levels of PGC1a during catabolic conditions (either in transgenic mice or by transfecting adult myofibers) spares muscle mass during denervation, fasting, heart failure, aging and sarcopenia—similar to the effect observed with expression of constitutively active FoxO3 (Sandri et al., 2006; Wenz et al., 2009). The positive action on muscle mass is due to the inhibition of autophagy-lysosome and UPS. PGC1a lowers protein breakdown by inhibiting the transcriptional activity of FoxO3, but it does not affect protein synthesis. These cofactors prevent the excessive activation of proteolytic systems by inhibiting the action of the pro-atrophy transcription factors without perturbing the translational machinery.

Working in concert with UPS signaling is the myostatin pathway. By binding to ActRIIB, myostatin activates type-1 activin receptor serine kinases, ALK4 or ALK5, which phosphorylate Smads 2 and -3 (key biomarkers) to exert changes in gene transcription (above figure). Notably, other TGF-b family members (e.g., activin-A and GDF11) can bind to ActRIIB and stimulate the same intracellular signaling pathway (Sandri, 2002, 2008; Sartori et al., 2009). To measure pathways regulating muscle atrophy and protein syntheses, the following key mRNA biomarkers were assayed: 1) Atrophy: MSTN, Atrogin-1, MuRF1, FoxO1, FoxO3, ALK4, Smad 1, Smad 3; 2) Anabolic: IGF1R, PGCIa, PGC1b, mitochondrial-FAM.

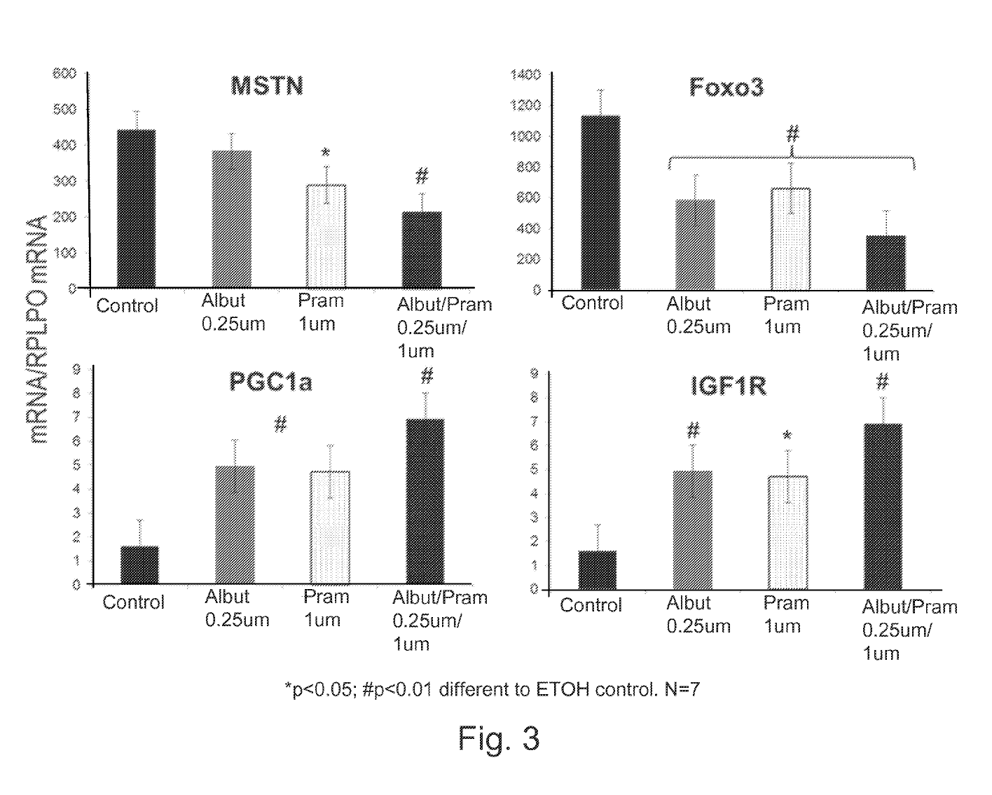

All RMS13 cells were treated for 24 hours with single or combination doses (n=10 each group). Surprisingly, analysis of the myoblast transcriptome from RMS13 cells treated with albuterol alone (0.25 mm), pramlintide (1 mm) or P/A; 1 mm/0.25 μm demonstrated no change in Atrogin-1, MuRF1, FoxOI, ALK4, Smad 1, Smad 3 or the mitochondrial anabolic factors PGC1b and TFAM mRNA under any treatment condition (data not shown). However, there was a strong trend for down regulation in P/A; 1 um/0.25 μm treated cells for MuRF1 (34±7 vs. 22±5, p=0.068; PBS control vs. P/A; 1 mm/0.25 μm respectively) and Smad3 (91±9 vs. 73±9, p=0.055; PBS control vs. P/A; 1 mm/0.25 μm respectively). Consistent with the anabolic role of amylin, pramlintide (1 mm) significantly lowered the mRNA expression of myostatin, FoxO3 and up regulated the expression of PGC1a and IGF1R (FIG. 3, all p<0.05). Similarly, albuterol (0.25 mm) down regulated FoxO3 mRNA and up regulated the expression of PGC1a and IGF1R (FIG. 3, all p<0.05); however it had no effect on myostatin expression, suggesting the beta-2 adreno-receptors agonist does not act on myostatin directly. Importantly, the greatest down regulation in myostatin and FoxO3 mRNA and up regulation in PGC1a and IGF1R mRNA occurred in the P/A; 1 mm/0.25 μm treated cells (FIG. 3, all p<0.01). Collectively, these data show that the combination of an amylin mimetic and a beta-2 adreno-agonist displays superior anabolic biomarker profile compared to single-dosed parent agent or PBS-control.

### Example 4—Insulin Provides Minimal Improvement on Catabolic Biomarkers

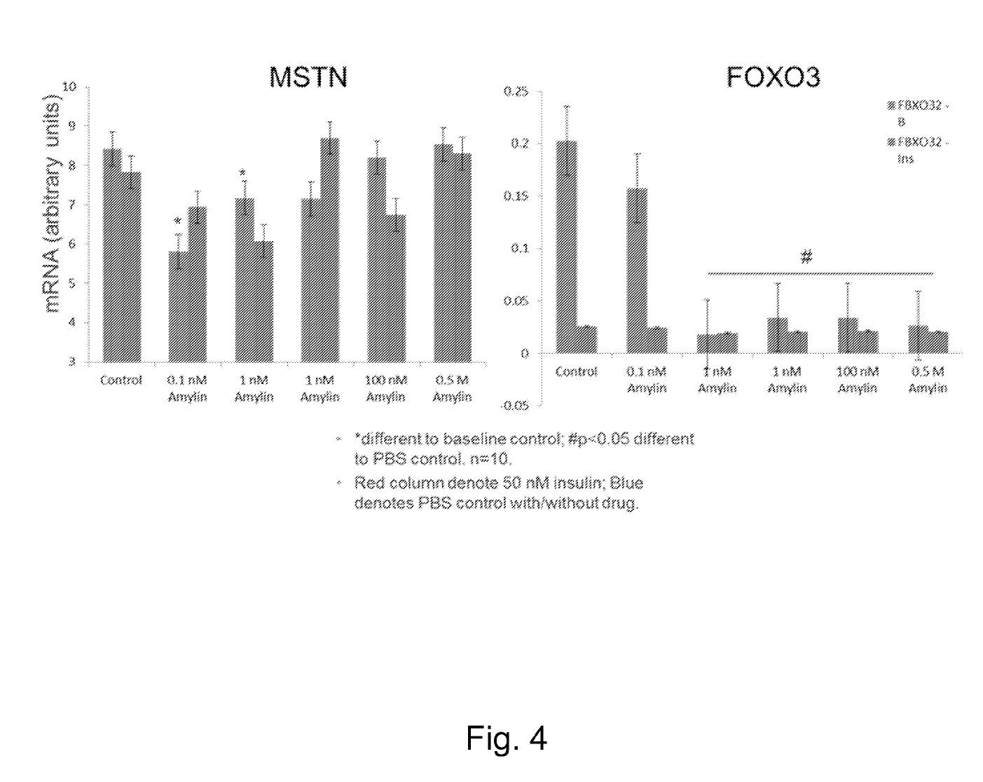

Pramlintide is chiefly used as an adjunct insulin mimetic in type 1 and type 2 diabetes to help manage glycemia. The action of the two hormones complements one another to better control glucose metabolism and energy balance. We investigated whether pramlintide with or without insulin could modulate myostatin (key biomarker) mRNA. As shown in FIG. 4, co-incubation with insulin (at a concentration typically used in glucose uptake assays) provided no additive benefit. This negative result provides important rationale for both preclinical and clinical study designs (see below). Amylin is thought to function as a neuroendocrine hormone (Young, 1997) and has been shown over many decades to have an effect of lowering food intake. When given intracerebroventricularly (icv), amyl in reduces food intake and body weight in rodents in a dose-dependent manner (Rushing et al., 2001). However, this does not preclude the use of pramlintide as an anabolic and body composition modifying agent that improves the lean-to-fat-tissue ratio in mammals. Data by Roth et al. demonstrate amylin's ability to increase muscle mass while at the same time dissipating WAT levels (Roth et al., 2006). Amylin is thought to regulate energy balance by inhibiting postprandial glucagon secretion and slowing the rate of nutrient delivery to the small intestine via an effect on gastric emptying. The rate of gastric emptying also plays a major role in blood glucose homeostasis in normal subjects by controlling the delivery of carbohydrate to the small intestine and the regulation of body weight (Reda et al., 2002). It has been proposed that amylin's effect on gastric emptying may be exerted via a central mechanism involving specific binding sites in the area postrema of the brain stem (Cline et al., 2008), and that the effect is mediated by the vagus nerve rather than a direct action on the stomach and digestion (D'Este et al., 1995; Maev and Dicheva, 2003). We have previously shown that the use of modified-pharmaceutical grade cellulose is able to dramatically slow the rate of gastric emptying and improve the lean-to-fat ratio in mice (Islam et al., 2012).

We hypothesize the role of amylin in mammalian physiology is to counteract the general anabolic effects of insulin (on all tissues including WAT) to favor a lean muscle and lower fat phenotype.

Given insulin is typically released in response to food intake and amylin has been shown to lower food intake, we propose a study design (preclinical and clinical) where pramlintide is dosed several hours before a meal. Pramlintide T-½-life is ˜45 min (Pencek et al., 2010; Weyer et al., 2001). Theoretically, dosing pramlintide 2-3 hours before a meal should allow pramlintide pharmacology to bypass a reduction in food intake (CNS-driven), while potentially providing the beneficial anabolic effects in the peripheral tissues. This rationale was tested in all preclinical studies with superior efficacy (see below).

### Example 5—Combination Therapy Directly Lowers Myostatin Secretion in Human RMS13 Cell Culture

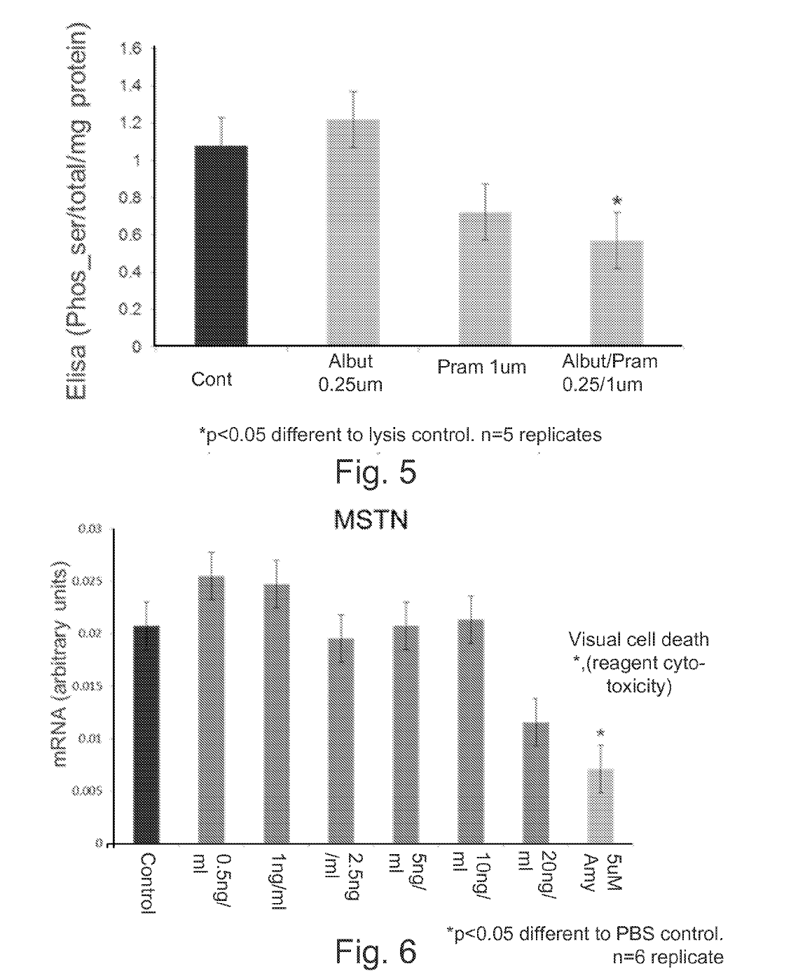

We demonstrated in FIG. 3 that pramlintide is able to directly lower myostatin mRNA in RMS13 myoblast. Myostatin's main sites of actions are muscle and bone in a catabolic fashion. Both IGF1 and amylin have roles in regulating osteoblast-bone proliferation (Cornish et al., 2004; Cornish and Naot, 2002) and muscle mass. These data could support the hypothesis that amylin's chief role in mammalian physiology is to regulate lean-to-fat ratio, in part by counteracting myostatin function. Although not intending to be bound by theory, we hypothesize that amylin alone and in combination with albuterol is able to directly suppress myostatin secretion in myoblasts. As shown in FIG. 5, treatment of myoblast with pramlintide (1 mm; p=0.061) and P/A; 1 mm/0.25 mm (p<0.05) lowered myostatin secretion into myoblast culture media (FIG. 5). The greatest suppression in hormone secretion however, was observed at P/A; 1 mm/0.25 mm.

### Example 6—Pramlintide Anabolic Specificity; Calcitonin does not Regulate Myostatin mRNA in RMS13 Cells

Calcitonin can be used therapeutically for the treatment of hypercalcemia or osteoporosis. Both calcitonin and amylin bind the same trimetric-calcitonin-ramp-receptor. An increased incidence of pituitary adenomas has been reported in rats given synthetic salmon calcitonin for 1 year. Calcitonin may be used diagnostically as a tumor marker for medullary thyroid cancer; also, elevated levels after surgery may indicate recurrence. Calcitonin may even be assayed in biopsy samples from suspicious lesions (e.g., lymph nodes that are swollen) to establish whether they are a metastasis of the original cancer (Bruera and Fainsinger, 1997; Busquets et al., 2012b). We next examined the dose-dependence relationship between the concentration of calcitonin and myostatin mRNA expression. As shown in FIG. 6, calcitonin did not modulate myostatin mRNA, while pramlintide (5 mm, p<0.05) was able to lower myostatin mRNA (FIG. 6). These data indicate the suppression of myostatin is specific to amylin biology.

### In Vitro Pharmacology Summary

Regarding in vitro pharmacology, Pramlintide is able to lower myostatin expression and secretion in RMS13 cells. This suppression is enhanced in the presence of albuterol. Combination treatment is able to increase anabolic and decrease catabolic markers in muscle cell system. Improvements in anabolic biomarker profile are independent of insulin or calcitonin. Therefore, the experiments described demonstrate that the function of amylin (or agonist) in mammalian physiology is to counteract the general anabolic effects of insulin and the catabolic drive of myostatin to favor a lean muscle and lower fat-phenotype. In vivo, combination therapy improves body composition in healthy, young B6 mice after 8-weeks.

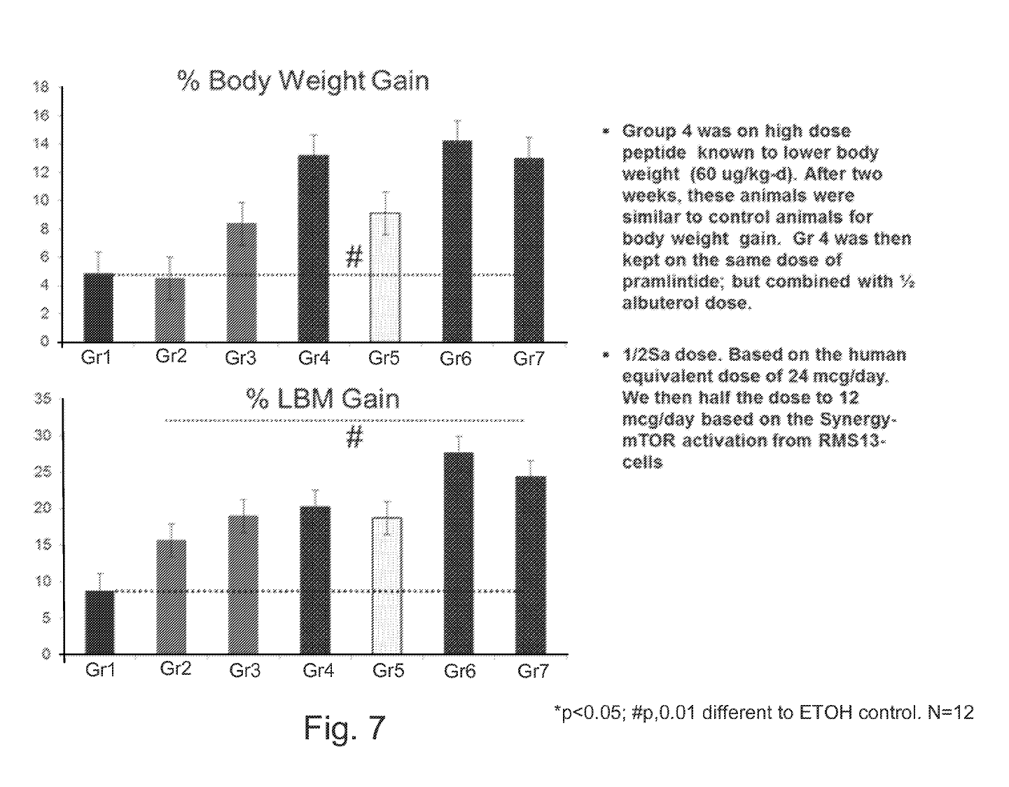

### In Vivo Study 1 (FIGS. 7-10)

Based on our in vitro experiments, we hypothesized that we could synergize muscle gain and improved biology with a combination of low doses of amylin (or agonist) and albuterol (or agonist). This study determined the effect of daily peripheral administration of pramlintide for 2-months on body weight, body composition and food intake in healthy, elderly mice.

Also was n=12 or n=24? T (here are two contradictory statements in this section) Treatment groups (n=24 all groups; n=12 current study 1): Note: n=24 for all groups is preferred. However, n=12 will likely be used in study-1. Another n=12 will likely stop at 4-weeks and assessment of BAT will be performed in Study 4 and 5—see last section.

### Procedure

Murine subjects were divided into seven treatment groups (total n=12): Group 1) SC 225 ml vehicle (PBS); Group 2) SC 15 mg/kg/mouse of pramlintide; Group 3) SC 30 mg/kg/mouse of pramlintide; Group 4) SC 60 mg/kg/mouse of pramlintide; Group 5) Albuterol 0.250 mg/kg. SC (2×0.125 mg/kg); Group 6) Albuterol 0.250 mg/kg. SC (2×0.125 mg/kg)+SC 15 mg/kg/mouse of pramlintide; Group 7) Albuterol 0.250 mg/kg. SC (2×0.125 mg/kg)+SC 30 mg/kg/mouse of pramlintide.

### Dosage

The most common dose to reduce food intake by −30% (over a 4-hour test period) in 16-week old mice is 100 mg/kg/mouse/day of amylin/pramlintide. Amylin's ability to lower food intake is transient (<4-hours) and at the doses proposed is expected not to lower food intake significantly over this period. The goal was to obtain a therapeutic dose of amylin/pramlintide that provides a tonic-signal, not to change body composition by lowering food intake, but rather to increase protein synthesis. Doses used by the Roth study (Roth et al., 2006) were 300 mg/kg/rat/day (76 nmol/kg·d). The average sizes of the rats used in the Roth study were 350 g. The average sizes of the 12-month mice used in this study were estimated to be 30 g. By scaling the dosing from rats from the Roth study to mice, our therapeutic dose was expected to be ˜30 mg/kg/mouse/day. The doses can be optimized by routine experimentation. There were three treatment groups with the following doses:

1. low (at half the expected therapeutic dose; 15 mg/kg/mouse/day);

2. target (30 mg/kg/mouse/day): and

3. high (60 mg/kg/mouse/day).

The potential for amylin resistance on food intake is high in mice as they enter late adulthood; therefore we included a high dose-group in the study design. The high-dose group is an important component, as it was not previously known if amylin resistance (lack of effect on satiety in elderly mice) also translates to protein-synthesis resistance. The latter factor is a complex question, as the trimeric receptor complex that binds amylin/pramlintide has different components, and therefore has different ED50 between the CNS and muscle.

A common low-dose of albuterol used in literature is BID-injections of 0.125 mg/kg, SC (total daily administration of 0.250 mg/kg, SC). Other beta(2)-adrenoceptor agonists such as formometerol (an albuterol isomer) have been used by other investigators in mice at similar doses proposed in these studies (Harcourt et al., 2007). Therefore, 0.125 mg/kg, SC (total daily administration of 0.250 mg/kg, SC) was the target dose. Our hypothesis was that by combining low-dose amylin and albuterol, we would observe similar body mass gains relative to target or high dose single-treated groups.

### Study Procedure

The mice were housed in single cages with a 12:12 light cycle, and allowed ad libitum access to food and water throughout the experiment. On day-1 food was weighed and an amount sufficient to last weekly intervals was provided. For the first two-weeks, food intake and body weight were monitored daily and thereafter biweekly for the remainder of the experiment. Body composition by NMR-spectroscopy was the primary endpoint, measured at Day-1 and at the end of the protocol (8-weeks). At Day-5, the mice were randomized to treatment and control groups, such that each group had the same mean body weight. Mice were allowed to acclimate for 5-days to being the sole mouse in the cage. Each day mice were dosed 1 hour after lights on, and the 2nd dosing two hours prior to lights out when mice initiate feeding. Pramlintide was taken directly from the FDA-approved Symlin drug (pramlintide acetate) obtained through the Pennington Biomedical Research Center (PBRC) pharmacy. Final injection volumes were 225 ml for vehicle or drugs. Symlin was supplied as a sterile drug at 0.6 mg/ml. All doses were adjusted according to this concentration.

As shown in FIG. 7, the combined doses of albuterol 12 μg/d/pramlintide-15 μg/kg/d and albuterol 12 mg/d/pramlintide 30 mg/kg/d gave more than an additive gain in muscle mass (lean mass) and loss of body fat over the treatment period, but this synergism was only statistically significant in the albuterol 12 mg/pramlintide-15 μg/kg/day group (data not shown). Statistical improvements in muscle gain and body fat reduction were also observed in 30 and 60 mg/kg/day pramlintide* and 24 mg/day albuterol groups (data not shown). [*Group 4, 60 mg/kg/day cohort—high dose group.] We initially monitored (first 7-days) food intake and body weight of the animals for stability (within 15% body weight loss) relative to the control group. We noticed Group 4 animals began to drop body weight (−5%) within the first week. We then added 12 μg of albuterol (Similar to Group 6 and 7) on top of the existing pramlintide dose. This change in pharmacology was adequate to reverse weight loss, and provided statistically significant increases in muscle mass by week-4. Gains were less than those in groups 6 and 7, suggesting a bell-shape curve for body weight gain as a function of increasing dose of pramlintide.

### Results

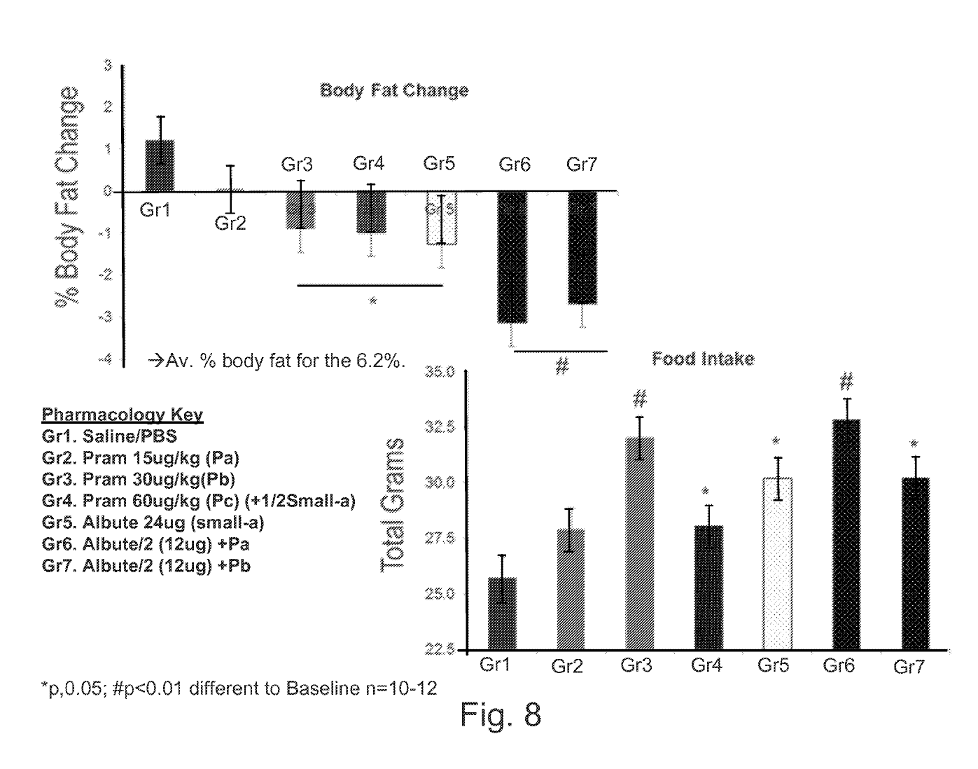

Over 8 weeks of treatment, the pramlintide/albuterol groups gained weight (17% and 15% of their initial body weight), similar to what was seen for the pramlintide 30 mg/kg/day and 60 mg/kg/day groups as well as the albuterol 24 mg/day groups; and greater than seen in the pramlintide 15 mg/kg/day and the saline groups (FIG. 7—top panel; P<0.05). However, the pramlintide/albuterol (albuterol 12 mg/pramlintide-15 ug/kg/day) group lost statistically more fat than was seen with a combination of the pramlintide/albuterol at higher doses of pramlintide but the same dose of pramlintide 30 ug/kg/day+12mg/day albuterol or 60 mg/kg/day+12 mg/day albuterol (Group 4) (FIG. 8—top panel, p<0.01), or with any of the mono-therapy groups (p<0.05).

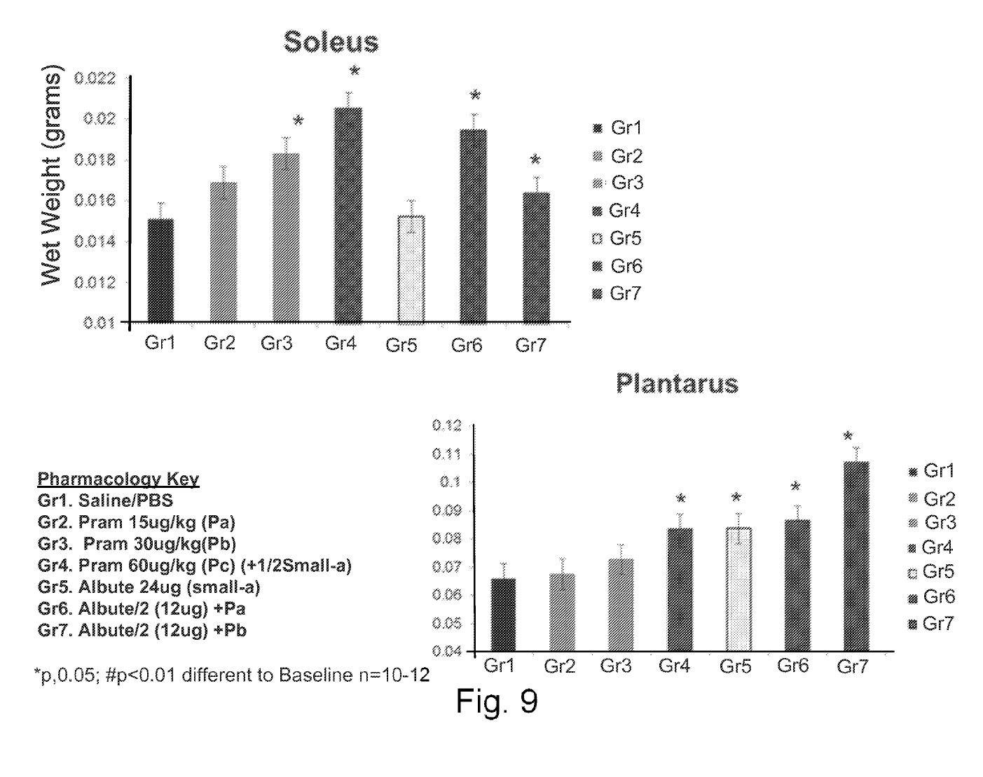

The loss in body fat for the albuterol 12 mg/pramlintide-15 mg/kg/day group was more than additive relative to the saline group (FIG. 8—top panel; p<0.001). The pramlintide/albuterol combination at a ratio of 15 mg/kg/d:12 mg/d produced a synergistic decrease in body fat, as compared to the additive effect of its individual components (FIG. 8—top panel, p<0.001). Lean tissue over the same period increased in all groups (p<0.05), except for the pramlintide 15 mg/kg/day and saline groups (FIG. 7—bottom panel). However, the increase in lean tissue for the combination pramlintide/albuterol (15 mg/kg/day+12 μg/day albuterol) group was greater than the sum of the increase in lean tissue in the monotherapy pramlintide and albuterol groups as compared to the saline control. This showed that pramlintide/albuterol (15 mg/kg/day+12 mg/day albuterol) gave a synergistic increase in lean tissue compared to the addition to its components (FIG. 7—bottom panel, p<0.001). We also examined the muscle mass of the soleus and plantarius muscle after tissue dissection. As shown FIG. 9, both pramlintide/albuterol had the ability to increase muscle-wet weight (p<0.05). When pramlintide, an amylin-mimetic, and albuterol, a beta-2 adrenergic agonist are combined, they provide a greater than additive improvement in body composition compared to the single-arm parent drugs.

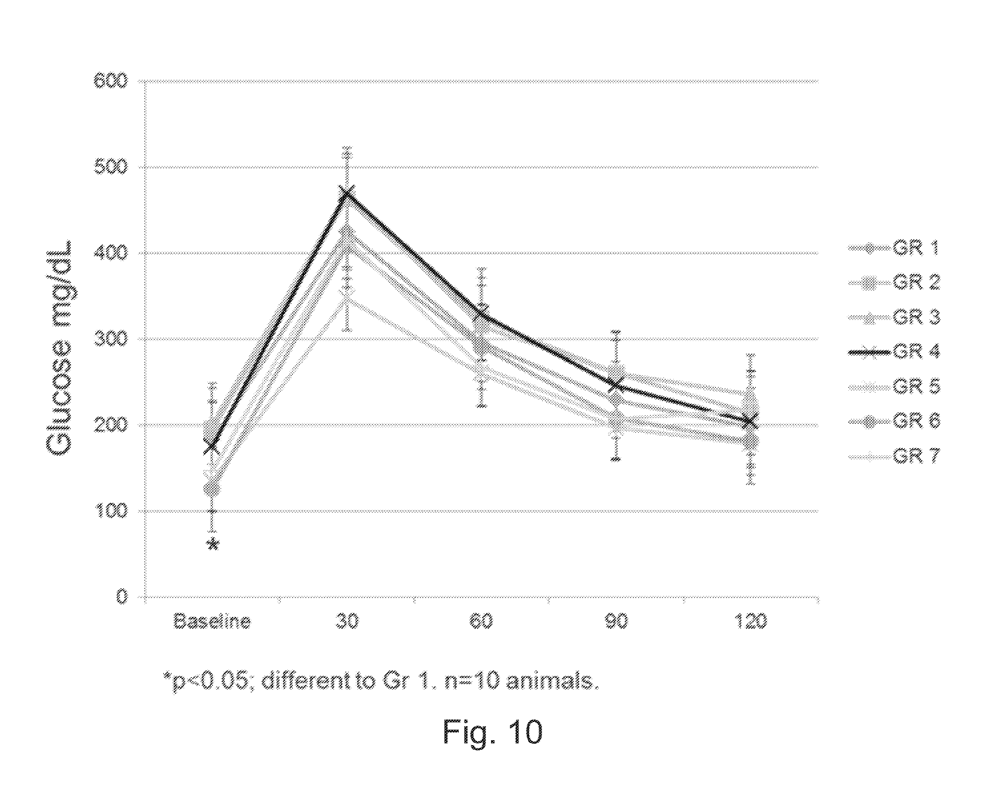

We next assessed whether amylin dosed prior to meals acts as an anorectic agent, as measured by cumulative food intake by rodents. (See generally Young et al., 1995; Young et al., 1996). As shown in FIG. 8 (bottom panel), by adopting a B.I.D dosing regimen several hours before meals (morning dosing/and ˜2 h before lights out), the pramlintide/albuterol groups that displayed the greatest improvements in muscle mass (FIG. 7—bottom panel) also demonstrated a significant increase in food intake over the study period (FIG. 8—bottom panel, p<0.05). This increase in food intake was associated with improved fasting glucose profile in the pramlintide 15 mg/kg/day+12 μg/day albuterol groups (FIG. 10, p<0.05) and is consistent with the well-known gluco-regulatory properties of pramlintide (Hoogwerf, 2006).

These data demonstrated the anabolic and or exegenic properties of the pramlintide/albuterol combination to improve body composition in healthy adult rodents. Since there is currently no pharmaceutical therapy with an FDA-approved indication to improve body composition in humans, a medication increasing lean tissue and decreasing fat tissue would fill an unmet medical need.

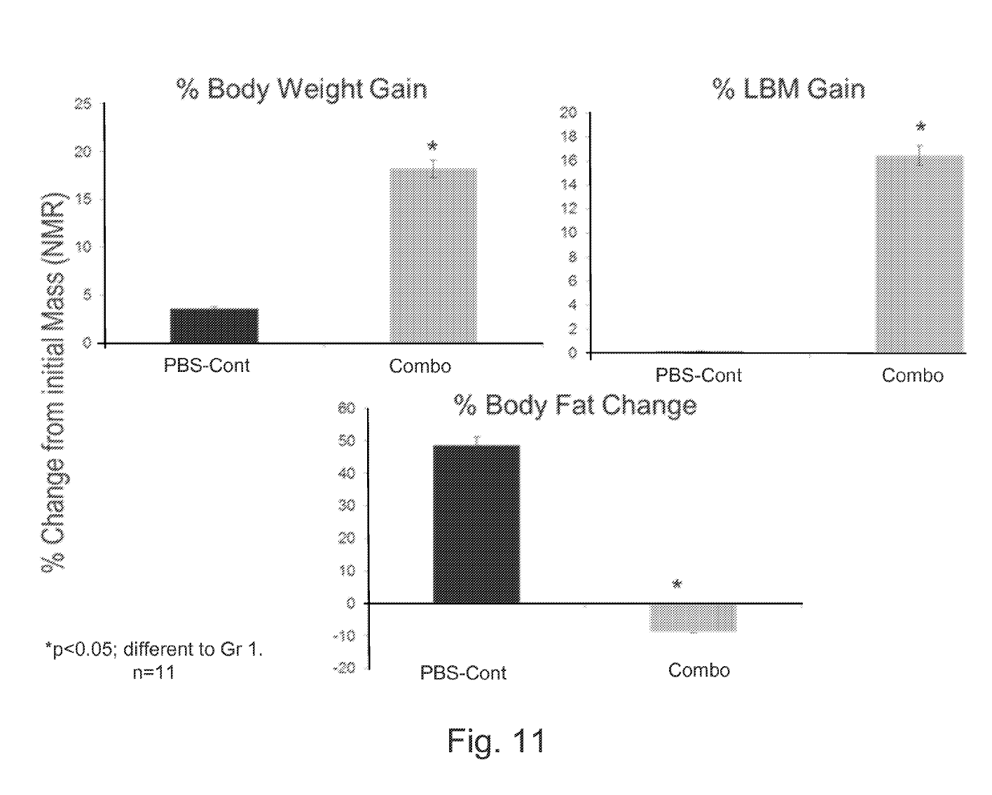

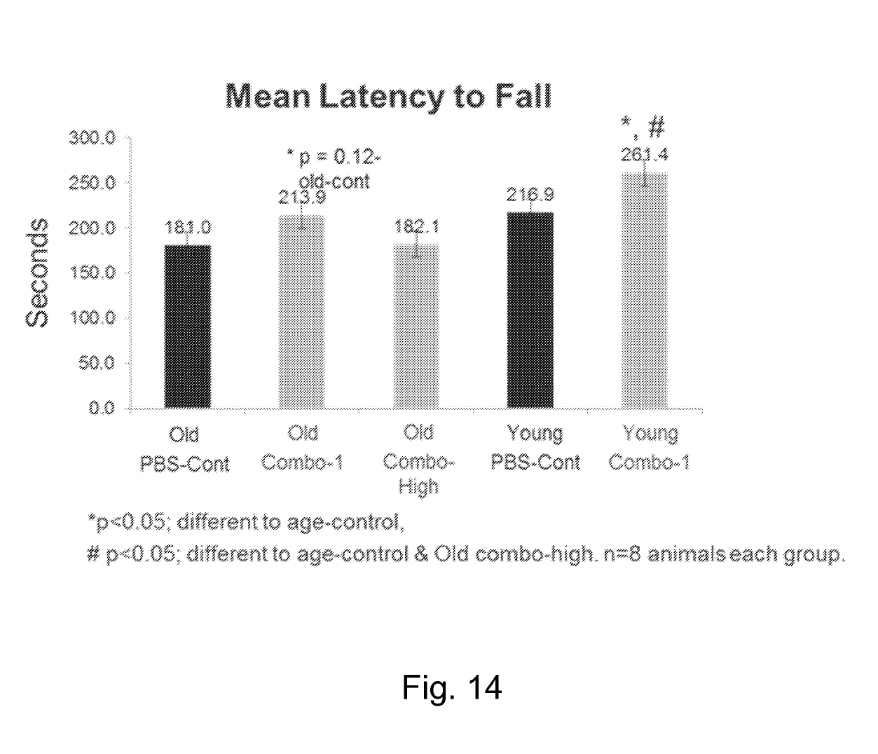

### In Vivo Pharmacology Studies 2 and 3 Combined (FIGS. 11-14)

Sarcopenia is prevalent in older people. The disease involves losses in muscle fiber quantity, quality, protein synthesis rates and anabolic hormone production. Sarcopenia leads to a decrease in overall physical functioning; it increases frailty, falls, and loss of independent living, and can even cause deaths (Hairi et al., 2010; Ward, 2011). It was estimated in 2004 that the annual US healthcare costs related to sarcopenia were over $20 billion per year (Zacker, 2006). It has been estimated that people lose 1%-2% of muscle annually after the age of 50, and 3% annually after the age of 60 (Hairi et al., 2010; Ward, 2011). This loss of muscle is accompanied by an increase in fat such that body weight stays approximately unchanged. Currently there are no therapeutic options for sarcopenia. The current set of in vivo experiments repeated In Vivo Study 1 in young-adult animals (control and combination group only) over a 5-month period to determine whether the efficacy of the pramlintide/albuterol was sustained (no development of drug resistance) over a significant life-period of a mouse (Study 2). These mice also served as control animals for Study 3. Study 3 investigated the efficacy of the pramlintide/albuterol combination in elderly mice (52-weeks+) over a 5-month treatment period (Study 3). Both studies investigated the functional significance of improved muscle mass with the pramlintide/albuterol combination.

### Procedures

Murine subjects were divided into 5 treatment groups (n=11 each group): Group 1) SC 225 ml vehicle (PBS), young-12-week old mice; Group 2) Albuterol 12 mg. SC+SC 15 mg/kg/mouse of pramlintide, young-12-week old mice; Group 3) SC 225 ml vehicle (PBS), 12-month old elderly mice; Group 4) Albuterol 12 mg. SC+SC 15 mg/kg/mouse of pramlintide, 12-month old elderly mice; Group 5) Albuterol 12 mg. SC+SC 60 mcg/kg/mouse of pramlintide, 12-month old elderly mice.

### Dosage

In Study 1, we had observed optimal efficacy with albuterol 12 mcg/pramlintide-15 mg/kg/day dose. This dose was therefore used in this study, and the same dose was given to both elderly and young mice allocated to the various treatments. The younger mice served as study controls. The potential for amylin resistance in elderly mice is known, as amylin's effects on satiety/food intake lessens in aging. Therefore we included a 12 mg/albuterol-60 mcg/kg/day of pramlintide dose-group to address potential of amylin resistance in aging. This higher dose was only given to elderly mice, as we did not observe efficacy at this dose in our earlier studies.

### Study Procedure

The mice were housed in single cages with a 12:12 light cycle, and allowed ad libitum access to food and water throughout the experiment. On day 1 food was weighed and an amount sufficient to last weekly intervals was provided. For the first week, food intake and body weight were monitored daily, and for the remainder of the experiment were monitored weekly. Body composition by NMR-spectroscopy was assayed at Day-land at the end of the protocol (20-weeks). Rotarod (Med-Associates, St Albans, Vt.) was used to at the same endpoints to assay motor function and coordination. This machine comprised a slowly accelerating rod, progressing from 4 to 40 rpm during a period of 300 seconds, and the time to fall was recorded for each animal. Mice were given 3 trials per day, with an inter-trial interval of 60 min. Each trial lasted a maximum of 360 seconds.

At Day 5, the mice were randomized to treatment and control groups, such that each group had the same mean body weight. Mice were allowed to acclimate for 5-days to being the sole mouse in a cage. On each day during the 4-week study period, animals were given the drug 1 hour after lights on, and 3 hours prior to lights out. Each mouse was weighed and injected SC with pramlintide/albuterol or 225 ml vehicle (PBS). Pramlintide was taken directly from the FDA-approved Symlin drug (pramlintide acetate) obtained through the PBRC pharmacy. Final injection volumes were 225 ml for vehicle or drug. Symlin was supplied as a sterile drug at 0.6 mg/ml as all doses were adjusted according to this concentration.

### Results

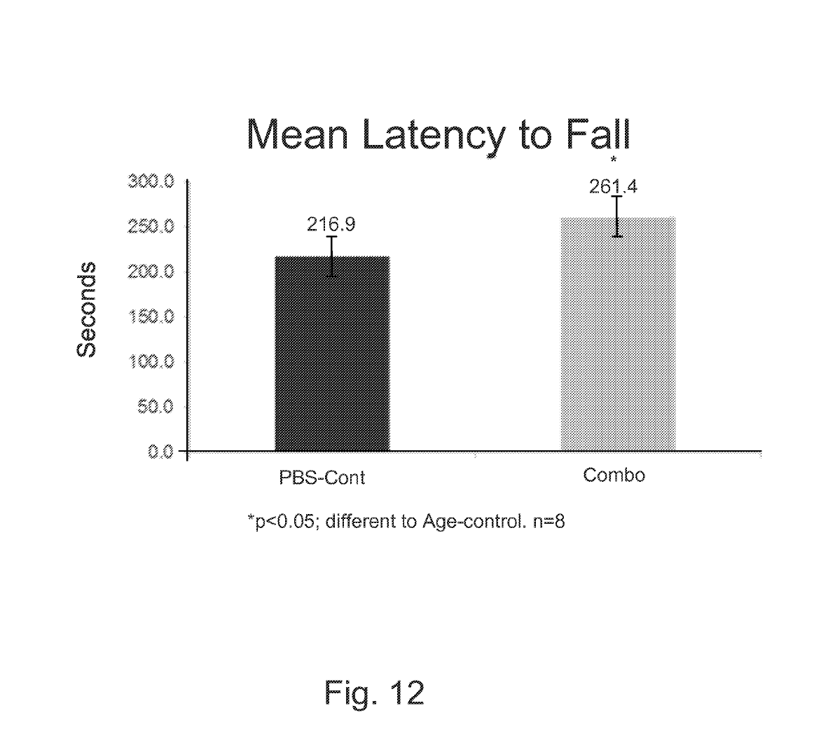

As shown in FIG. 11, 5 months of 12 mg. SC+SC 15 mg/kg albuterol/pramlintide therapy resulted in a change of approximately 18% body weight, 17% muscle mass and an approximate reduction of 48% WAT from their initial starting points (FIG. 11, p<0.01; combo-1). The changes relative to PBS controls were greater than synergistic (3-fold+) (FIG. 11, p<0.01). In addition, when the young animals were tested for functionality, the 12 mg. SC+SC 15 mg/kg albuterol/pramlintide displayed significantly improved exercise performance relative to PBS controls (FIG. 12, p<0.05; combo-high). These data highlight several key findings, 1) the combination of albuterol/pramlintide yielded therapeutic efficacy at least up to 5 months of therapy, 2) synergetic improvements in body composition were comparable to Study 1 (over a 2-month duration). This indicated the ratio of 12 mg SC+SC 15 mg/kg albuterol/pramlintide used in both studies was preferred for the current formulation and 3) improvements in muscle mass observed in albuterol/pramlintide group translated into improved physical performance that may display beneficial utility in the elderly.

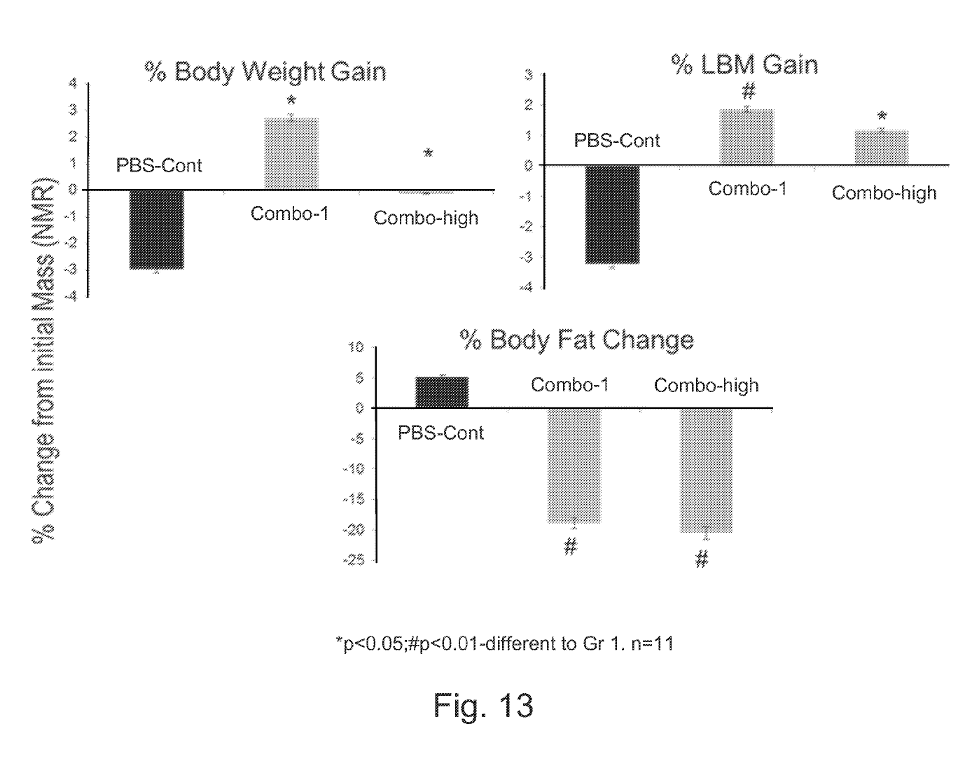

Surprisingly, we did not observe the same magnitude of change in body composition in elderly mice. As shown in FIG. 13, 5 months of 12 mg SC+SC 15 mg/kg albuterol/pramlintide therapy resulted in a change of approximately 3% body weight, 1.75% muscle mass and an approximate reduction of 20% WAT from their initial starting points (FIG. 13, p<0.01). The 12 mg SC+SC 60 mg/kg albuterol/pramlintide did not change body weight over the 5-month period. However, there was a small but significant change in muscle mass, approximately 1.25%, and an impressive reduction of approximately 20% WAT (FIG. 13, p<0.01). As aging progresses, mammals normally lose muscle mass and replace this with WAT. The elderly PBS-control group displayed a change of approximately a 3% decrease in body weight, approximately a 3% decrease in muscle mass and approximately a 5% increase in WAT from their initial starting points (FIG. 13, p<0.01). The changes in body weight, muscle mass and WAT in the 12 mg SC+SC 15 mg/kg albuterol/pramlintide group relative to PBS controls were more than additive (FIG. 11, p<0.01). Collectively, these data indicated that the combination of 12 mg. SC+SC 15 mg/kg albuterol/pramlintide was able to modestly improve body composition in elderly mice over a 5-month study period. However, relative to untreated elderly animals, the combination of albuterol/pramlintide was effective in inhibiting the natural decrease in muscle mass and increase in WAT so widely seen in aging.

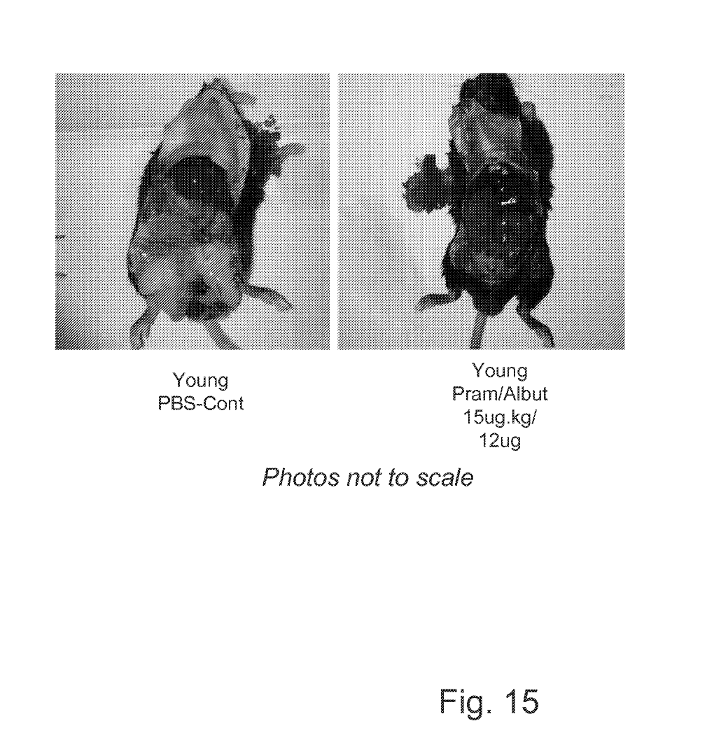

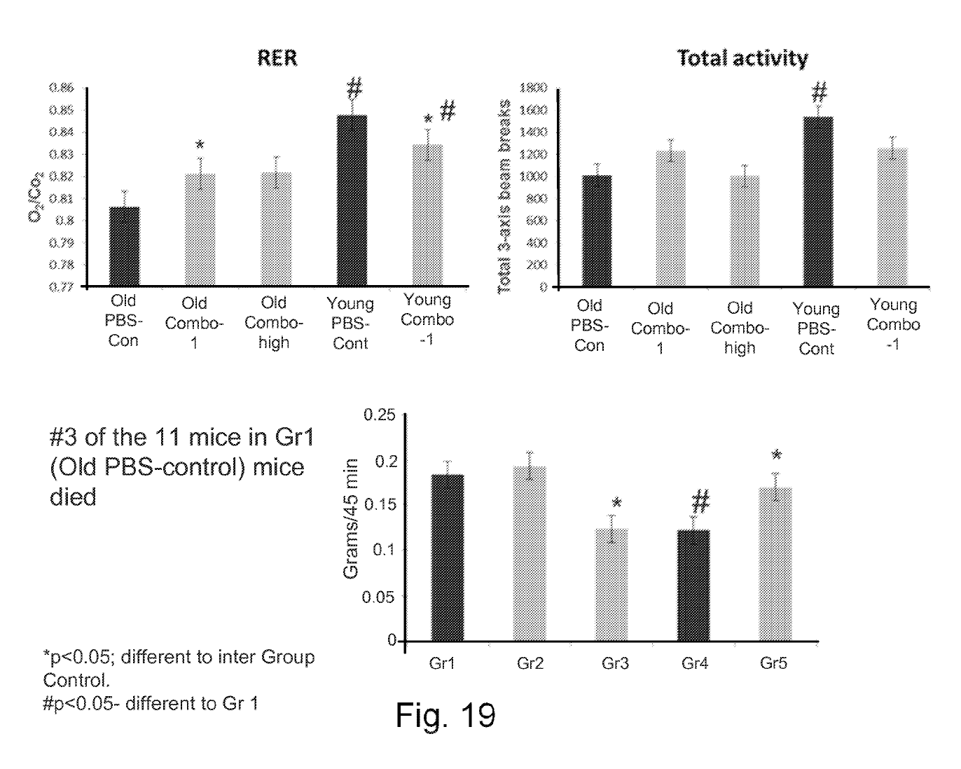

### In Vivo Pharmacology Studies #4 and #5 (FIGS. 15-20)

Obesity is a condition of excess levels of adipose tissue. Its associated disorders are common and pose a serious public health problem in the United States and throughout the world. Upper body adiposity is the strongest risk factor known for type 2 diabetes mellitus, and is also a strong risk factor for cardiovascular disease. Obesity also includes factors that make up metabolic syndrome and other comorbidities such as hypertension, atherosclerosis, congestive heart failure, stroke, gallbladder disease, osteoarthritis, sleep apnea, reproductive disorders such as polycystic ovarian syndrome, and some cancers. In addition to lowering life span and posing a serious risk of the co-morbidities listed above, obesity also causes an increased risk of illness in other disorders such as infections, varicose veins, acanthosis nigricans, eczema, exercise intolerance, insulin resistance, hypertension hypercholesterolemia, cholelithiasis, orthopedic injury, and thromboembolic disease (Rissanen et al., 1990). The worldwide medical cost of obesity and associated disorders is enormous.

The pathogenesis of obesity is multifactorial. One factor is that, in obese subjects, nutrient availability and energy expenditure do not come into balance until there is excess adipose tissue. To lower the level of excess adiposity, individuals may try to modulate food intake or increase energy expenditure. Low calorie diets and exercise are common routes of therapy, along with a few pharmacological agents that modulate satiety and either inhibit the absorption of nutrients or lower food intake. Currently, there are no therapies on the market that physicians can prescribe for patients to regulate energy expenditure.

Increasing muscle mass to combat muscle wasting will also elevate the ability to burn fat and lower WAT levels; however, this is not the only mechanism to burn excess WAT. WAT and BAT are characterized by different anatomical locations, morphological structures, functions, and regulations. WAT and BAT are both involved in energy balance. WAT is mainly involved in the storage and mobilization of energy in the form of triglycerides, whereas BAT specializes in dissipating energy as heat during cold- or diet-induced thermogenesis. In the BAT literature, this is termed as uncoupling the energy transfer (of electrons that normally are used to make ATP in mitochondria), to instead dissipate excess energy (WAT) as heat (“wasting energy”) via the uncoupling protein 1 (UCP1) molecule (Matthias et al., 1999; Rial et al., 1998; Shabalina et al., 2008). In Studies 1 and 2, we demonstrated that the combination of pramlintide and albuterol provided a greater improvement in body composition (increase in muscle mass and decrease in fat mass) compared to the monotherapy parent drugs dosed at higher concentrations and relative to placebo (saline). Part of this fat loss may be due to the formation of BAT, given that these combination therapy groups had elevated food intake despite highly and synergistic decreases in WAT.

Another factor influencing the loss of muscle protein is impaired activation and proliferation of muscle progenitor or satellite cells. Both muscle and BAT tissues are highly thermogenic, suggesting a common metabolic profile. At the molecular level, differentiation into WAT, BAT or beige adipocytes (WAT that act like BAT) have distinct mechanisms (Seale et al., 2008). Previously, WAT and BAT were thought to be derived from the same precursor cell. However, recent studies demonstrated that BAT shares a progenitor cell (Myf5+) with skeletal muscle, and not with white adipocytes (Bostrom et al., 2012; Kajimura et al., 2009; Ohno et al., 2012; Seale et al., 2008). Although not intending to be bound by theory, we hypothesize that a drug combination that has the utility in increasing muscle mass and proliferation may also have the same ability to increase BAT levels. The Myf5+ precursors are induced to transform into mature brown adipocytes by bone morphogenetic protein 7 (BMP7), peroxisome proliferator-activated receptor-y (PPAR-y) and CCAAT/enhancer-binding proteins (C/EBPs) in cooperation with the transcriptional co-regulator PR domain-containing 16 (PRDM16) and PGC-1a (Kajimura et al., 2009; Seale et al., 2007). White adipocytes can also be transformed to brown-like adipocytes, called beige/brite adipocytes, by cold exposure, a B-adrenerqic agonist or a PPAR-y agonist, AR (adrenergic receptor), FGF21 (Fibroblast growth factor 21) and PGC-1a enhanced (as seen with pramlintide treatment in RMS13 cells) expression. Furthermore, expression of PRDM16 increases the amount of both BAT content and the bona-fide biomarker of BAT activity, UCP1 protein content (Kajimura et al., 2009; Seale et al., 2007; Uldry et al., 2006).

Although not intending to be bound by theory, we hypothesize that the combined use of amylin (pramlintide) (or agonist) and albuterol (or agonist) will 1) will synergistically increase the content of BAT, PRDM16 and UCP1 in young mice and 2) protect and maintain a high core body temperature in elderly and young mice when exposed to a thermal 24-hour-4-degree Celsius challenge. Analogous effects are expected in other mammals, including humans.

### Example 7

### Procedure

Murine subjects were divided into seven treatment groups (n=12 each group): Group 1) SC 225 ml vehicle (PBS); Group 2) SC 15 μg/kg/mouse of pramlintide; Group 3) SC 30 mg/kg/mouse of pramlintide; Group 4) SC 60 mg/kg/mouse of pramlintide; Group 5) Albuterol 0.250 mg/kg. SC (2×0.125 mg/kg); Group 6) Albuterol 0.250 mg/kg. SC (2×0.125 mg/kg)+SC 15 mg/kg/mouse of pramlintide; Group 7) Albuterol 0.250 mg/kg. SC (2×0.125 mg/kg)+SC 30 mg/kg/mouse of pramlintide.

### Procedure

Murine subjects were divided into five treatment groups (n=11 each group): Group 1) SC 225 ml vehicle (PBS), young-12-week old mice; Group 2) Albuterol 12 meg. SC+SC 15 mg/kg/mouse of pramlintide, young-12-week old mice; Group 3) SC 225 ml vehicle (PBS), 12-month old elderly mice; Group 4) Albuterol 12 mg. SC+SC 15 mg/kg/mouse of pramlintide, 12-month old elderly mice; Group 4) Albuterol 12 mg. SC+SC 60 mcg/kg/mouse of pramlintide, 12-month old elderly mice.

### Procedure

The experiments were conducted in two phases. Phase 1 was a 1-month treatment to induce changes in body composition and formation of BAT in combination-treated young animals. If this proved to be effective in forming BAT in young animals, we would then proceed to the next phase. Phase 2 was to determine whether the increases in BAT content translated into elevated core body temperature and energy expenditure when mice were exposed to a thermal 4° C. challenge. Elevated core body temperature and energy expenditure are two bona-fide biomarkers of BAT function.

### Phase 1

The mice were housed in single cages with a 12:12 light cycle (Groups 1-7), and allowed ad libitum access to food and water throughout the experiment. On day 1 food was weighed and an amount sufficient to last weekly intervals was provided. For the first week, food intake and body weight was monitored daily and thereafter for the remainder of the experiment, was monitored weekly. Body composition by NMR-spectroscopy was determined at Day 1. At Day 5, the mice were randomized to treatment and control groups, such that each group had the same mean body weight. Mice were allowed to acclimate for 5 days to being the sole mouse in the cage. On each day during the 4-week study period, animals were given drug 1 hour after lights on and 3 hours prior to lights out. Each mouse was weighed and injected SC with pramlintide/albuterol or 225 ml vehicle (PBS). Pramlintide was taken directly from the FDA-approved Symlin drug (pramlintide acetate) obtained through the PBRC pharmacy. Final injection volumes were 225 ml for vehicle or drug. Symlin was supplied as a sterile drug at 0.6 mg/ml as all doses were adjusted according to this concentration.

### Phase 2

At the end of the Phase 1 treatment period, mice continued pharmacotherapy for an additional 2 weeks until the end of the indirect calorimetry-thermal challenge. During this period, all 55 mice (Groups 1-5) were given adaptation trials (2-weeks) to expose animals to the metabolic cage system. Body composition by NMR was assessed at end of Phase 1 (B-above/start of Phase 2) and again at 6 weeks (C-above/end of Phase 2) in all mice. Indirect calorimetry was conducted at 4° C. thermal challenge for 24 hours at 6-weeks (C-above/end of Phase 2).

### Results

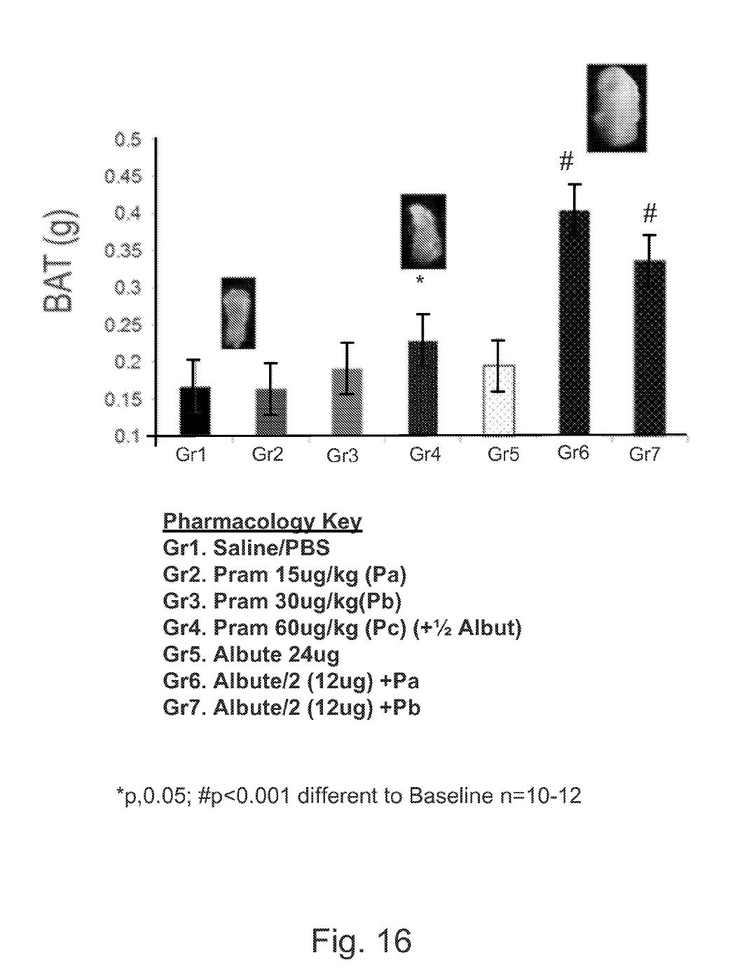

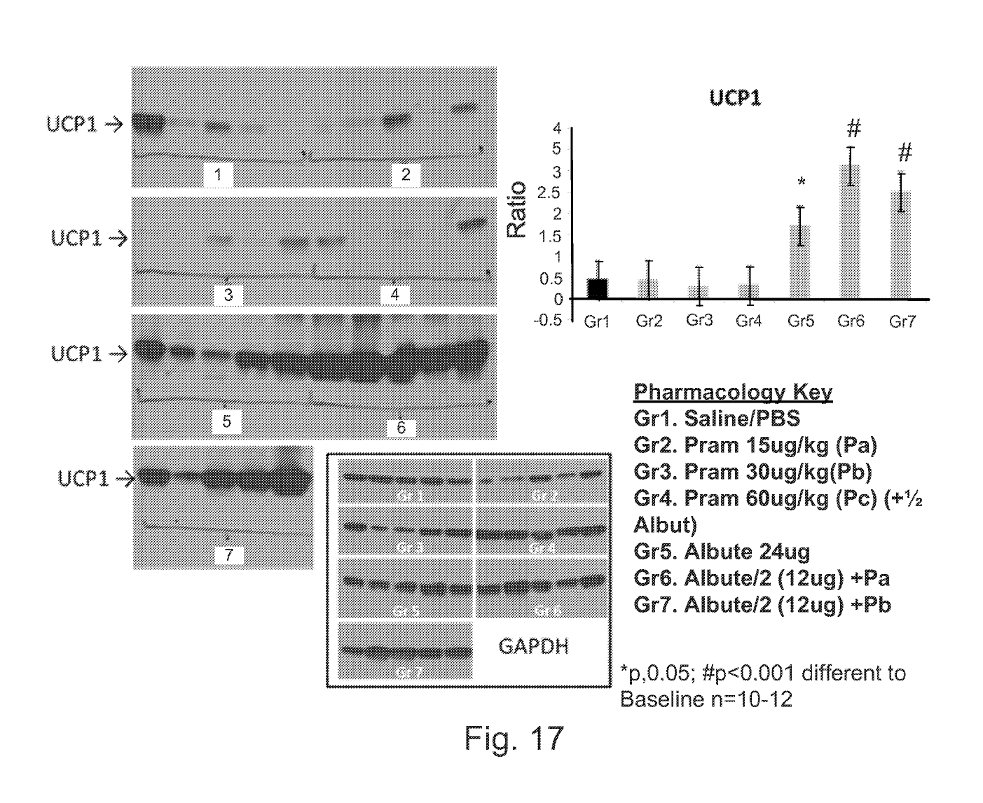

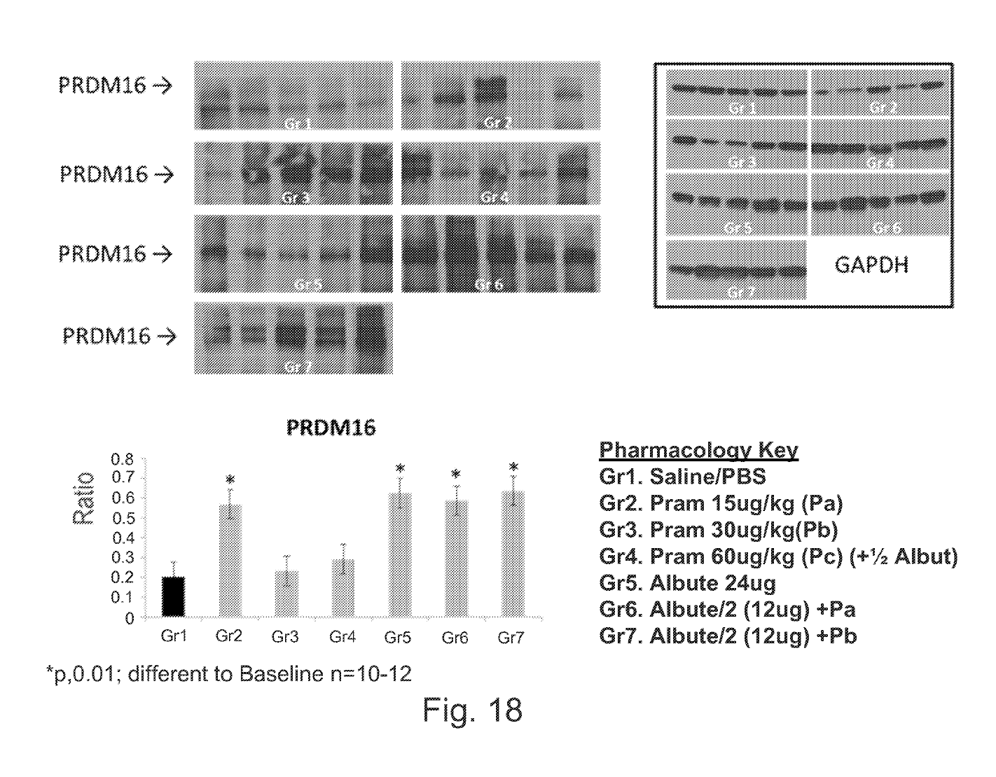

As shown in FIG. 15, the combination of 12 mg+15 mcg/kg SC albuterol/pramlintide therapy induced a dramatic lean phenotype and general loss of WAT (FIG. 8—top panel), despite increased food intake (FIG. 8—bottom panel), suggesting increased energy expenditure. Analysis of BAT content in all treatment groups revealed significant increases in all combination groups (albuterol 12 mg/pramlintide-15 μg/kg/day; albuterol 12 mg/pramlintide-30 mg/kg/day and albuterol 12 μg/pramlintide-60 mg/kg/day, FIG. 16, all p<0.05). However, the pramlintide/albuterol (albuterol 12 mg/pramlintide-15 μg/kg/day) group gained statistically more BAT than the combination combinations with higher doses of pramlintide combined with the same dose of albuterol 30 mg/kg/day+12 mg/day albuterol and 60 mg/kg/day+12mg/day albuterol (Group 4) (FIG. 16, p<0.01), or any of the monotherapy treatments (p<0.05). This increase in BAT for the albuterol 12 mg/pramlintide-15 μg/kg/day group was more than additive relative to the saline group (FIG. 8—top panel; p<0.001). This shows that pramlintide/albuterol given at the ratio of 15 mg/kg/d:12 μg/d generated a synergistic increase in BAT compared to the sum of its individual components (FIG. 16, p<0.01). Consistently, both combination groups displayed an increase in PRDM16 transcription factor protein expression in BAT (FIG. 18, p<0.01) tissue. The 12 mg/pramlintide 15 mg/kg/day; albuterol 12 μg/pramlintide-30 mg/kg/day; and albuterol groups displayed a highly significant and synergistic increase in UCP1 protein expression (greater than 6-fold) after 4-weeks of therapy relative to PBS-controls (FIG. 17, p<0.01). These data demonstrated the combination of pramlintide and albuterol is able to synergistically increase the content of BAT and the key functional biomarker UCP1 protein expression.

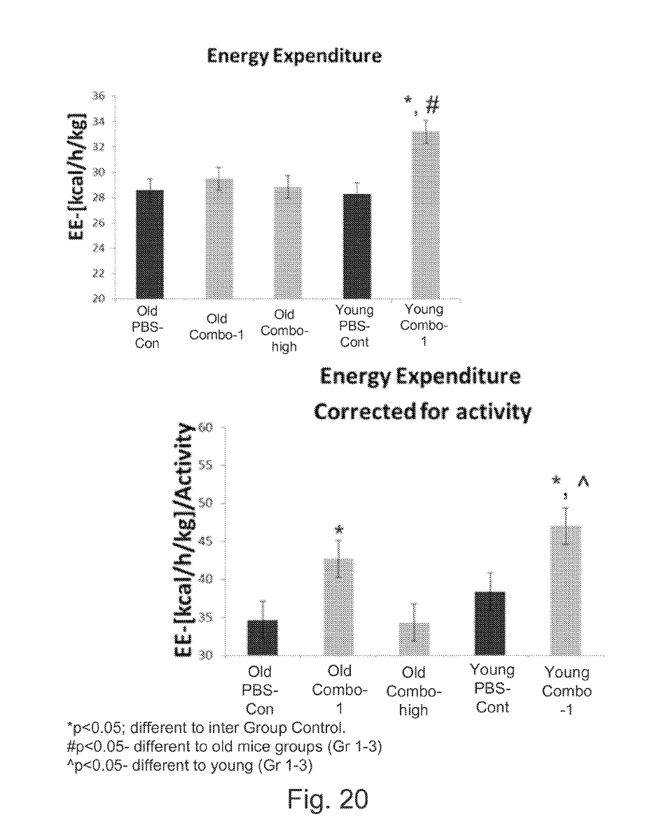

Although not intending to be bound by theory, we hypothesized that the combination of pramlintide/albuterol should also cause an increase in energy expenditure in response to a suitable stimulus. Cold exposure, similar to hibernation, is able to increase sympathetic nervous system activity and BAT activity. As seen in FIG. 20, a dose of albuterol 12 μg/pramlintide-15 mg/kg/day increased energy expenditure in young animals only (Group 5, FIG. 20, p<0.05). This induction was in accordance with the Phase 1-group 6 data described above, in which 12 mg/pramlintide-15 mg/kg/day produced the greatest increase in BAT content. After correcting for covariates of body weight and activity levels, the dose of albuterol 12 μg/pramlintide-15 mg/kg/day produced a significant increase in energy expenditure in both young and elderly animals (FIG. 20, right-panel; p<0.05).

The combination of albuterol 12 mg/pramlintide-15 mg/kg/day dramatically increased the formation of BAT, and UCP1 protein, and energy expenditure—all of which are associated with increase in lean muscle and a reduction in adiposity.

### In Vivo Pharmacology Summary

Combination treatment synergistically lowered % body fat in young mice after 8-16-weeks. Weight gain increased (20-30%) and synergistically elevated lean muscle mass (15%+) in combination groups in young animals. We could directly regulate myostatin levels as well as increase protein synthesis via mTOR activation. Treatment groups had an elevation in food intake, indicating a negative fat balance and an increase in energy expenditure. Cold exposure elevated 24-EE, independent of “BW” and activity. Combination treatment dramatically increased BAT content and key BAT-activity biomarkers UCP1 and PRDM16. The increased BAT activity was associated with synergistic deceases in WAT. Combination treatment synergistically lowered % body fat in elderly mice after 20 weeks. Combination treatment increased body weight and muscle mass gains in elderly mice after 20 weeks. The improvements in body composition were modest relative to what we observed in younger animals. However, the combination of pramlintide and/albuterol did inhibit or reverse the natural regression in body composition (low muscle mass/increased fat mass) so widely observed in ageing mammals.

### Example 8

### The Anabolic Properties of the Amylin Hormone

Cachexia and sarcopenia are frequent and overlapping conditions in older people. Both conditions are characterized by protein catabolism, apoptosis, inflammation, cachexia, insulin resistance and death. The ability of the elderly or patients with critical illness (i.e. pulmonary disease, chronic HIV infection/muscle wasting, spinal cord injury or many other medical conditions that limit muscle use) to perform exercise and combat muscle wasting is limited. Therefore, the identification of medicines and novel pathways modulating both cachexia and sarcopenia; independent of exercise may hold promise for treatment of muscle atrophy conditions. Amylin is a 37-amino acid peptide that is synthesized in pancreatic (3-cells and co-secreted with insulin in response to nutrient ingestion. A large body of data shows that Amylin is an important glucoregulatory hormone because it complements the action of insulin in maintaining glucose homeostasis during the postprandial period. Roth et al., has previously examined the effects of amylin and pair feeding (PF) on body weight and metabolic parameters in diet-induced, obesity-prone rats (Roth et al., 2006). Surprisingly, Roth and colleagues demonstrated a disproportionate increase in lean body mass and protein synthesis in amylin-treated DIO rats compared with PF animals (with improved insulin sensitivity) or untreated controls after 22 days. The amylin pathway has pro-anabolic effects in skeletal muscle. In addition, pharmacological inhibition of amylin receptor in osteoblast, abolishes IGF-1 ability (anabolic hormone) to promote bone growth indicating an important role for amylin biology in growth and regeneration. Importantly, myostatln has been shown to principally work at the level of muscle and bone to regress the active mass of those tissues. Loss of muscle mass is a serious consequence of many diseases. It can lead to weakness, fragility, loss of independence and increased risk of comorbidities including but not limited to insulin resistance, chronic inflammation, cachexia and death. There is no product available for the treatment of patients with muscle wasting and its co-morbidities and muscle dystrophy.

Amylin complements the action of insulin in maintaining glucose homeostasis during the postprandial period. Like insulin, amylin is completely absent in individuals with Type I diabetes. Pramlatide is an analogue of amylin marketed in the USA as the FDA-approved drug Symlin (Pramlatide Sulfate). Pramlatide has been approved by the FDA, for use by Type 1 and Type 2 Diabetics who use insulin and allows patients to use less insulin; lowers average blood sugar levels, and substantially reduces what otherwise would be a large unhealthy rise in blood sugar that occurs in diabetics right after eating. Apart from insulin analogs, pramlatide is the only drug approved by the FDA to lower blood sugar in type 1 diabetics since insulin in the early 1920s.

Reduction in HbA1c and weight loss (in some studies) has been shown in insulin-treated patients with type 2 diabetes taking pramlatide as an adjunctive therapy. Pramlatide MOA is multi-facet: aids in the absorption of glucose by slowing gastric emptying, promoting satiety via hypothalamic receptors (different receptors than for GLP-1), and inhibiting inappropriate secretion of glucagon, a catabolic hormone that opposes the effects of insulin and amylin. Pramlintide also has effects in raising the acute first-phase insulin response threshold following a meal. However, the monotherapy use of Amylin, does not cause hypoglycemia events and not all studies have shown weight loss. Along with data by Roth et al. and in osteoblasts mentioned above, these data raise the possibility that amylin may have other functions in the human body.

Data from our laboratory demonstrates a down regulation of myostatin (catabolic hormone & the downstream transcription factors FoxO-1/-3) mRNA and an elevation of pro-anabolic targets (PGC1a, calcineurin mRNA and AkT and mTOR-activity) after treating human myoblast for 24 h with pramlintide (FDA-approved amylin-mimetic). Although not intending to be bound by theory, we hypothesize the amylin pathway has a role in the regulation of muscle mass in mammals and the use of pramlintide will result in improved body composition and increase muscle mass in healthy humans by lowering the production of myostatin and increasing protein synthesis via mTOR activation.

Salbutamol or albuterol is an inexpensive and safe short-acting b₂-adrenergic receptor agonist used for the relief of bronchospasm in conditions such as asthma and chronic obstructive pulmonary disease. It is marketed as Ventolin among other brand names and was approved by the FDA in the USA in 1968. Salbutamol has been shown to improve muscle weight in rats by lowering the breakdown of muscle mass. By combining salbutamol and Amylin at clinical used and/or lower doses, we hypothesized we will be able to synergize (more than double) lean body mass gains by: Inhibiting the level of myostatin and thus function; decreasing the transcriptional activity of signaling proteins downstream of myostatin; decreasing the level and function of key protein involved in muscle break down; and increasing the activity of mTOR/protein synthesis.

In a recent pharmacology assessment in young, healthy BL-male mice, we completed a 7-week placebo controlled (Gr 1) study with treatment-arms that included the use of pramlintide alone at low (Gr 2. 15 mg/kg); mid (Gr 3. 30 mg/kg) and high (Gr 4. 60 mg/kg) or Salbutamol alone (Gr 5. 24 mg) or combined with Pramlintide (Gr 6. 15 mg/kg+12 mg of Salbutamol; Gr 7. 30 mg/kg+12 mg of Salbutamol). After the treatment period, groups 3-7 showed significant increases in bodyweight from baseline and relative to placebo control; while lean body mass (LBM) was significantly increased in all treatment-arms, indicating a specific effect on muscle mass. Importantly, groups 6 and 7 demonstrated a synergistic elevation in LBM gains relative to placebo group (−8% Increase in placebo relative to −30% in Gr 6 and 7).

### Summary

Amylin and salbutamol alone or in combination (in vitro) are able to increase the signaling proteins involved in muscle building and in vivo, this translated to a synergistic increase in lean body mass gains over placebo control. Part of the ability to increase muscle mass is attributed to a down regulation of androgens (proteins involved in muscle break-down). Importantly, by combining amylin and salbutamol, we are able to significant lower the therapeutic dose for efficacy over the use of the parent drug. All drug doses used in pharmacology studies are at clinically relevant doses.

### Example 9

The epidemic of obesity and excess body fat accumulation is tightly linked to the prevalence of insulin resistance, type 2 diabetes, dyslipidemia, CVD and other disorders. The role of white adipose tissue (WAT) is to store lipids, however in syndromes such as obesity and insulin resistance, WAT adipose tissue can be a source of immune “dysfunction” and chronic inflammation that is linked to disease etiology. Brown adipose tissue (BAT) on the other hand is the opposite and its prime function is to bum off excess energy in the form of heat. In the BAT science area, this is termed uncoupling the energy transfer (of electrons) to make ATP (energy currency of the cell), to instead dissipate excess energy (adipose tissue) as heat via the uncoupling protein 1 (UCP1) molecule. By combining amylin (pramlintide drug) and Beta-2 Receptor agonism (B2R) (Salbutamol drug) embodiments of the present disclosure can: synergistically lower excess body fat relative to non-treatment by specifically targeting the fat cell; synergistically preserve lean body mass by inducing anabolic pathways to target muscle building; Induce muscle building targets In skeletal muscle (PGC1a, mTFAM, IGF1R, mTOR activity) and lower the level of catabolic targets (MSTN, Activin Recptor Iib/Ib, Foxo1/3 Smad3); specially target HSL by increasing activity and flow of free fatty acids from WAT and lowering LPL-activity; and slow the rate of re-uptake into WAT, which allows for target organs such as skeletal muscle and BAT to use FFA as fuel and in turn lowering excess body fat; synergistically increase the formation of beige and brown adipocytes; and synergistically increase the expression of key BAT factors (UCP1, PGC1a, Cidea, PRDM16, Pax3). The combination of amylin and B2R-agonism can be a method and practice for 1) inducing lean body mass gains; 2) increase brown adipose tissue formation/UCP1 content; 3) improve body composition profile by lowering adiposity and be used for the prevention of excess adiposity, metabolic disease and its' co-morbidities.

### Summary of In Vivo Methods Used in the Experiments

### Drugs Used

Pharmaceutical grade albuterol sulfate is available either as an inhaler for a direct effect on bronchial smooth muscle, or in pill form. It is not practical to dose mice with an inhaler, nor is it possible to accurately dose with the pill form. An injectable pharmaceutical grade albuterol is not commercially available. Therefore a research grade product of the highest purity available (98%) was used. Albuterol (98% pure, Sigma Aldrich) was dissolved in sterile saline and filter-sterilized through a 0.21-mm filter prior to use.

### 24-Hour Food Intake

Food intake in grams was measured manually each 24 hours. Body Composition was determined via NMR using a Bruker mini-spec system designed for rat or mouse. Animals were transported in microisolater covered cages to the designated room containing the NMR. Animals were placed in the designated tube, the tube was placed in the mini-spec device, and measurements were taken for approximately 2 minutes. The animals were then returned to their home cage.

### Rotarod Activity Test

Mice were given a habituation trial on day-2, in which they were placed on the Rotarod at a constant speed (4 rpm) for 60 s. The following day (Day-3) each mouse was given three trials during which the Rotarod started at 4 rpm and accelerated to 40 rpm over a period of five minutes or until the mouse fell off the Rotarod. No electrical stimulation or stimulus was given to the mice. Animal “performance” was based on duration on the Rotarod.

### 4° C. Thermal Challenge with Core Body Temperature Measure

One 24-hour thermal challenge was given to mice placed at 4° C. ambient temperature; rectal temperature was recorded every hour for 24 hours.

### REFERENCES

Allen, D. L., et al (2011) Med Sci Sports Exerc 43, 1828-1835.
Amoroso, P., et al (1993), Thorax 48, 882-885.
Argiles, J. M., et al (2012), Drug Discov Today 17, 702-709.
Batsis, J. A., et al (2014), Eur J Clin Nutr.
Bernardo, B. L., et al (2010), PLoS One 5, e11307.
Bodine, S. C., et al (2001), Science 294, 1704-1708.
Bostrom, P., et al (2012), Nature 481, 463-468.
Bruera, E., et al (1997), N Engl J Med 336, 962-963.
Busquets, S., et al (2012a), Oncol Lett 3, 185-189.
Busquets, S., et al (2012b), J Cachexia Sarcopenia Muscle 3, 37-43.
Calnan, D. R., et al (2008), Oncogene 27, 2276-2288.
Cline, M. A., et al (2008), Regul Pept 146, 140-146.
Colburn, W. A., et al (1996), J Clin Pharmacal 36, 13-24.
Collins-Hooper, et al (2014), J Gerontal A Bioi Sci Med Sci.
Cornish, J., et al (1995), Biochem Biophys Res Commun 207, 133-139.
Cornish, J., et al (1998), Am J Physiol 275, E694-699.
Cornish, J., et al (1999), J Bone Miner Res 14, 1302-1309.
Cornish, J., et al (2004), Biochem Biophys Res Commun 318, 240-246.
Cornish, J., et al (2002), Curr Pharm Des 8, 2009-2021.
D'Este, L., et al (1995), Arch Histol Cytol 58, 537-547.
Dimsdale, J. E., et al (1996), Hypertension 27, 1273-1276.
Fakhouri, T. H., et al (2012), NCHS Data Brief, 1-8.
Fearon, K., et al (2013), Nat Rev Clin Oncol 10, 90-99.
Fineman, M. S., et al (2002), Metabolism 51, 636-641.
Fujita, M., et al (2013), J Bioi Chem 288, 19593-19603.
Gedulin, et al (1997), Metabolism 46, 67-70.
Girgenrath, S., et al (2005), Muscle Nerve 31, 34-40.
Hairi, N. N., et al (2010), J Am Geriatr Soc 58, 2055-2062.
Han, H. Q., et al (2013), Int J Biochem Cell Bioi 45, 2333-2347.
Harcourt, L. J., et al (2007), Neuromuscul Disord 17, 47-55.
Hittel, D. S., et al (2009), Diabetes 58, 30-38.
Holz, M. K., et al (2005), J Bioi Chem 280, 26089-26093.
Hoogwerf, B. J. (2006), Cleve Clin J Med 73, 477-484.
Islam, A., et al (2012), Obesity (Silver Spring) 20, 349-355.
Kahn, S. E., et al (1998), Diabetes 47, 640-645.
Kajimura, S., et al (2009), Nature 460, 1154-1158.
Kamalakkannan, G., et al (2008), J Heart Lung Transplant 27, 457-461.
Kline, W. O., et al (2007), J Appl Physiol (1985) 102, 740-747.
Koda, J. E., et al (1992), Lancet 339, 1179-1180.
Lebrasseur, N. K. (2012), Diabetologia 55, 13-17.
Lee, S. J. (2004), Annu Rev Cell Dev Bioi 20, 61-86.
Maev, I. V., et al (2003), Eksp Klin Gastroenterol, 86-90, 120.
Magnuson, B., et al (2012), Biochem J 441, 1-21.
Matthias, A., et al (1999), J Bioi Chem 274, 28150-28160.
McCormick, C., et al (2010), J Appl Physiol (1985) 109, 1716-1727.
McPherron, A. C., et al (1997), Nature 387, 83-90.
McPherron, A. C., et al (1997), Proc Natl Acad Sci USA 94, 12457-12461.
Musaro, A., et al (2001), Nat Genet 27, 195-200.
Nojima, H., et al (2003), J Bioi Chem 278, 15461-15464.
Nyholm, B., et al (1998), Horm Metab Res 30, 206-212.
Nyholm, B., et al (1996), J Clin Endocrinol Metab 81, 1083-1089.
Ohno, H., et al (2012), Cell Metab 15, 395-404.
Otulakowski, G., et al (2007), Am J Respir Cell Mol Bioi 37, 457-466.
Park, J. J., et al (2006), Physiol Genomics 27, 114-121.
Pencek, R., et al (2010), Diabetes Obes Metab 12, 548-551.
Puigserver, P., et al (2003), Nature 423, 550-555.
Reda, T. K., et al (2002), Obes Res 10, 1087-1091.
Rial, E., et al (1998), Biofactors 8, 209-219.
Rigamonti, A. E., et al (2009), Horm Metab Res 41, 23-29.
Rissanen, A., et al (1990), Diabetes Res Clin Pract 10 Supp/1, S195-198.
Rommel, C., et al (2001), Nat Cell Biol 3, 1009-1013.
Roth, J. D., et al (2006), Endocrinology 147, 5855-5864.
Rushing, P. A., et al (2001), Endocrinology 142, 5035.
Sacheck, J. M., et al (2004), Am J Physiol Endocrinol Metab 287, E591-601.
Sandri, M. (2002), Curr Opin Clin Nutr Metab Care 5, 249-253.
Sandri, M. (2008), Physiology (Bethesda) 23, 160-170.
Sandri, M., et al (2006), Proc Natl Acad Sci USA 103, 16260-16265.
Sartori, R., et al (2009), Am J Physiol Cell Physiol 296, C1248-1257.
Schiaffino, M. V. (2010), Int J Biochem Cell Bioi 42, 1094-1104.
Schuelke, M., et al (2004), N Engl J Med 350, 2682-2688.
Schulze, P. C., et al (2005), Circ Res 97, 418-426.
Seale, P., et al (2008), Nature 454, 961-967.
Seale, P., et al (2007), Cell Metab 6, 38-54.
Seeman, E. (2004), Calcif Tissue Int 75, 100-109.
Shabalina, I. G., et al (2008), Biochim Biophys Acta 1777, 642-650.
Sharma, M., et al (1999), J Cell Physiol 180, 1-9.
Tisdale, M. J. (2009), Physiol Rev 89, 381-410.
Tremblay, F., et al (2001), J Bioi Chem 276, 38052-38060.
Trevaskis, J. L., et al (2010a), Obesity (Silver Spring) 18, 21-26.
Trevaskis, J. L., et al (2010b), Physiol Behav 100, 187-195.
Uldry, M., et al (2006), Cell Metab 3, 333-341.
Ward, J. (2011), Australas J Ageing 30, 61-62.
Welle, S., et al (2002), Exp Gerontal 37, 833-839.
Wenz, T., et al (2009), Proc Natl Acad Sci USA 106, 20405-20410.
Weyer, C., et al (2001), Curr Pharm Des 7, 1353-1373.
Yarasheski, K. E., et al (2002), J Nutr Health Aging 6, 343-348.
Young, A. A., et al (1995), Diabetologia 38, 642-648.
Young, A. A., et al (1996), Metabolism 45, 1-3.
Zacker, R. J. (2006), et al Jaapa 19, 24-29.
Zhou, Z. R., et al (2013), Theriogenology 79, 225-233.
Zito, C. I., et al (2007), J Bioi Chem 282, 6946-6953.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

1. A pharmaceutical composition comprising a therapeutically effective amount of an amylin receptor agonist or a pharmaceutically acceptable salt thereof and a therapeutically effective amount of a beta-2-adreno-receptor agonist or a pharmaceutically acceptable salt thereof, and a pharmaceutically acceptable carrier, to treat a condition.

2. The pharmaceutical composition of claim 1, wherein the condition is selected from the group consisting of: muscle wasting, sarcopenia, a muscle wasting-related disorder, a muscle wasting-related disease, a muscle wasting-related condition, diabetes, insulin-resistance syndrome, cancer-cachexia, COPD, AIDS, congestive heart failure, sepsis, anorexia, pulmonary disease, excess adiposity, an excess adiposity-related disorder, an excess adiposity-related disease, an excess adiposity-related condition, lypodystrophy, nonalcoholic steatohepatitis, a cardiovascular disease, polycystic ovary syndrome, metabolic syndrome, and a combination thereof.

3. The pharmaceutical composition of claim 1, wherein the amylin receptor agonist is an amylin-hormone mimetic.

4. The pharmaceutical composition of claim 3, wherein the amylin-hormone mimetic is pramlintide.

5. The pharmaceutical composition of claim 1, wherein the beta-2-adreno-receptor agonist is selected from the group consisting of: albuterol, an albuterol derivative, an albuterol agonist, and a combination thereof.

6. The pharmaceutical composition of claim 5, wherein the beta-2-adreno-receptor agonist is albuterol.

7. The pharmaceutical composition of claim 1, wherein the condition is to increase muscle mass, reduce fat mass, or a combination thereof with or without an increase in endurance.

8. A method of treating a condition, comprising: delivering to a subject in need thereof, a therapeutically effective amount of an amylin receptor agonist or a pharmaceutically acceptable salt thereof and a therapeutically effective amount of a beta-2-adreno-receptor agonist or a pharmaceutically acceptable salt thereof, and a pharmaceutically acceptable carrier.

9. The method of claim 8, wherein the condition is selected from the group consisting of: wherein the condition is selected from the group consisting of: muscle wasting, sarcopenia, a muscle wasting-related disorder, a muscle wasting-related disease, a muscle wasting-related condition, diabetes, insulin-resistance syndrome, cancer-cachexia, COPD, AIDS, congestive heart failure, sepsis, anorexia, pulmonary disease, excess adiposity, an excess adiposity-related disorder, an excess adiposity-related disease, an excess adiposity-related condition, lypodystrophy, nonalcoholic steatohepatitis, a cardiovascular disease, polycystic ovary syndrome, metabolic syndrome, and a combination thereof.

10. The method of claim 8, wherein the condition is to increase muscle mass, reduce fat mass, or a combination thereof with or without an increase in endurance.

11. (canceled)

12. (canceled)

13. (canceled)

14. A pharmaceutical composition comprising a therapeutically effective amount of at least two different anti-catabolic agents, and a pharmaceutically acceptable carrier, to treat a condition.

15. The pharmaceutical composition of claim 11, wherein a first anti-catabolic agent is selected from the group consisting of a pramlintide acetate amylin analog agonist, and a long-acting amylin analog agonist; and wherein a second anti-catabolic agent is selected from the group consisting of: a beta-2 receptor adreno-agonist, a beta-2 receptor adreno-agonist analog, a short acting beta-2 receptor adrenoagonist agonist, and a short acting adreno-agonist.

16. The pharmaceutical composition of claim 11, wherein a first anti-catabolic agent is selected from the group consisting of albuterol, an albuterol derivative, and an albuterol agonist; and wherein a second anti-catabolic agent is selected from the group consisting of: clenbuterol, a clenbuterol derivative, and a clenbuterol agonist.

17. The pharmaceutical composition of claim 11, wherein a first anti-catabolic agents is selected from the group consisting of: a myostatin antagonist, an ActIIR-antagonist, an activin-A antagonist, IGF1-agonist, IGF1-receptor-agonist, IGF1, a recombinant IGF1R, IGF1 derivative, albuterol, clenbuterol, an albuterol analog, a clenbuterol analog, an albuterol agonist, a clenbuterol agonist, amylin, an amylin analog, and an amylin agonist.

18. The pharmaceutical composition of claim 11, wherein a first anti-catabolic agent is selected from the group consisting of: amylin, an amylin analog, and an amylin agonist, and a second anti-catabolic agent selected from the group consisting of: a beta-2 receptor adreno-agonist, an adreno-agonist, a beta-2 receptor adreno-agonist analog.

19. (canceled)

20. (canceled)

21. (canceled)

22. (canceled)

23. (canceled)

24. (canceled)

25. A pharmaceutical composition comprising a therapeutically effective amount of at least two different anti-adiposity agents, and a pharmaceutically acceptable carrier, to treat a condition.

26. The pharmaceutical composition of claim 16, wherein a first anti-adiposity agent selected from the group consisting of: amylin, an amylin analog, and an amylin agonist; and a second anti-adiposity agent selected from the group consisting of: a beta-2 receptor adreno-agonist, and a beta-2 receptor adreno-agonist analog.

27. The pharmaceutical composition of claim 16, wherein a first anti-adiposity agent selected from the group consisting of: amylin, an amylin analog, and an amylin agonist; and a second anti-adiposity agent selected from the group consisting of: a beta-2 receptor adreno-agonist, and a beta-2 receptor adreno-agonist analog.

28. (canceled)

29. (canceled)

30. (canceled)
